(19) United States
(12) Patent Application Publication
(10) Pub. No.: US 2020/0369618 A1
(43) Pub. Date: Nov. 26, 2020

(54) ASYMMETRIC DERIVATIVES OF POLYPHENOLS OF THE DINAPHTHALINE SERIES, PROCESS OF PREPARING AND USE THEREOF

(71) Applicant: NEARMEDIC INTERNATIONAL LIMITED, Nicosia (CY)

(72) Inventors: Vladimir Georgievich NESTERENKO, Moscow (RU); Anatoly Petrovich SUSLOV, Moscow (RU); Sergey Alexandrovich TSYRULNIKOV, Moscow (RU); Mikhail Nikolaevich NENASHEV, Moscow (RU); Irina Vladislavovna KISELEVA, Moscow reg. (RU); Vitaly Vladimirovich KUZNETSOV, Moscow (RU)

(73) Assignee: NEARMEDIC INTERNATIONAL LIMITED, Nicosia (CY)

(21) Appl. No.: 16/958,618

(22) PCT Filed: Dec. 25, 2018
(86) PCT No.: PCT/RU2018/050169

§ 371 Date: Jun. 26, 2020

(30) Foreign Application Priority Data
Dec. 26, 2017 (RU) 2017145952

(51) Int. Cl. C07D 217/02; C07C 47/57; C07C 50/38; C07C 69/736; C07C 251/24; C07D 211/58; C07C 309/13; C07D 213/80; C07D 211/62; C07C 243/18

(52) U.S. Cl. CPC C07D 217/02; C07C 47/57; C07C 50/38; C07C 69/736; C07C 243/18; C07D 211/58; C07C 309/13; C07D 213/80; C07D 211/62; C07C 251/24

(57) ABSTRACT

The present invention relates to the field of organic chemistry and pharmacology and describes novel asymmetric derivatives of polyphenols of the dinaphthalene series of general Formula (I) or general Formula (II), a process of preparing thereof and use thereof as antiviral agents.

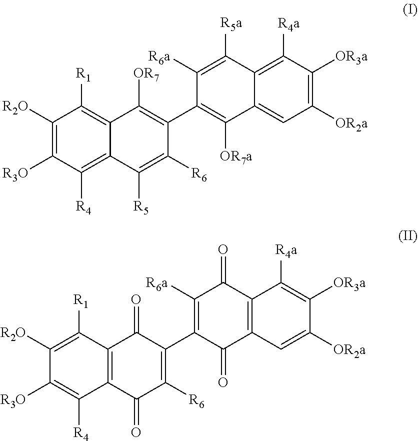

### TECHNICAL FIELD

The present invention relates to the field of organic chemistry and pharmacology and describes novel asymmetric derivatives of polyphenols of the dinaphthalene series, processes of preparing and use thereof.

### BACKGROUND ART

Over the long time, polyphenols of the dinaphthalene series have been the subject of multiple studies. Most publications in this field describe the natural polyphenol gossypol (1) and products of its synthetic modifications (apogossypol (2), gossypolone (3), apogossypolone (4)). These compounds have been studied for their antiviral (Biochem. Pharmacol., 1993, 46, 251-255), antitumor (Mol. Pharmacol., 1990, 37, 840-847), antimycotic and antioxidant (Biochem. Pharmacol., 1989, 38, 2859-2865) activities.

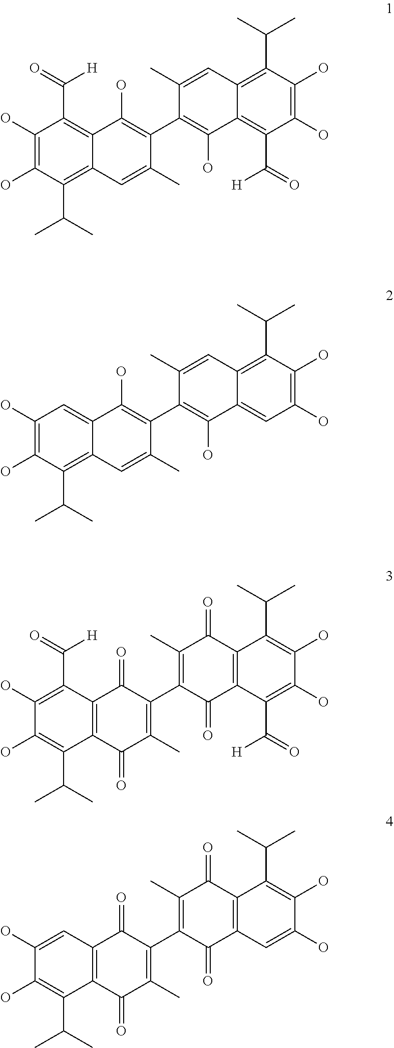

Beside the abovementioned compounds, many gossypol derivatives have been synthesized (J. Am. Oil Chem. Soc. 2006, 83, 4, 269-301), including products of its synthetic modification on the aldehyde groups (azomethine derivatives (5)) or phenol (alkyl ethers (6)), and products of its oxidation (gossypolone (7)) and alkaline destruction of the naphthalene ring (gossindan (8) etc.). The majority of these synthetic derivative products are characterized by the presence of two identical (symmetric) naphthalene or other fragments.

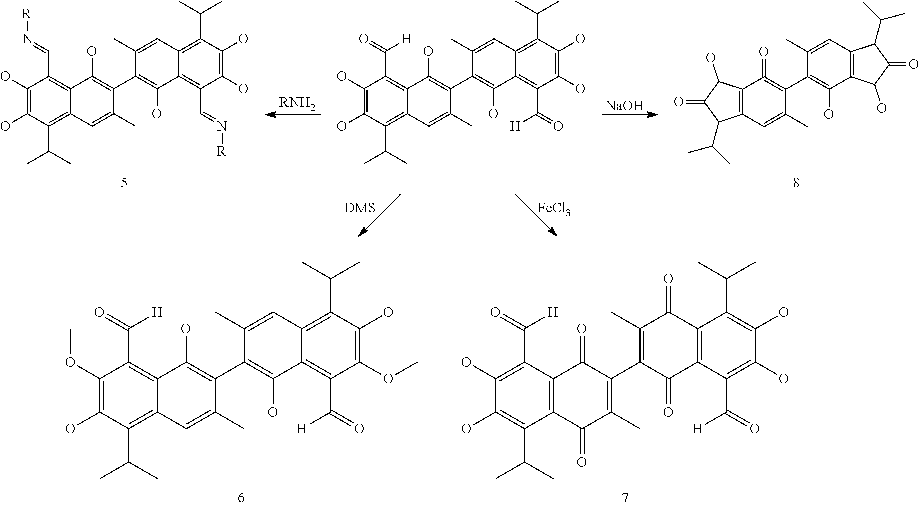

However, to date, relatively few polyphenols of the naphthalene series are known that contain two structurally different naphthalene fragments. The main directions of asymmetric gossypol derivate research have been reviewed by K. Z. Tilyabaev (Chemistry of plant raw materials, 2013, No. 3, 17-31). The most represented compounds in this field are asymmetric Schiff bases (9), as well as mono-azaderivatives, obtained through reaction with corresponding diazonium salts (10).

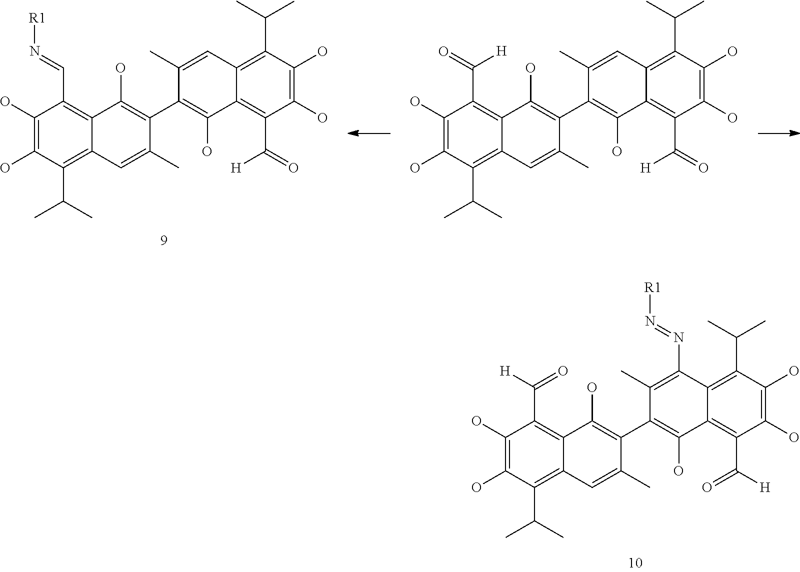

Additionally synthesized asymmetric gossypol derivatives include those obtained through a hydroxylic group modification (11, 12) (Chin. Chem. Lett., 1992, vol. 3, 165-166; Yin J. Chemical modification and biological activity exploration of the natural product- gossypol: PhD dissertation in Food technology, Graduate School of Clemson University, USA, 2010), as well as halogen derivatives (13) (J. Org. Chem., 1992, vol. 57, 2316-2320).

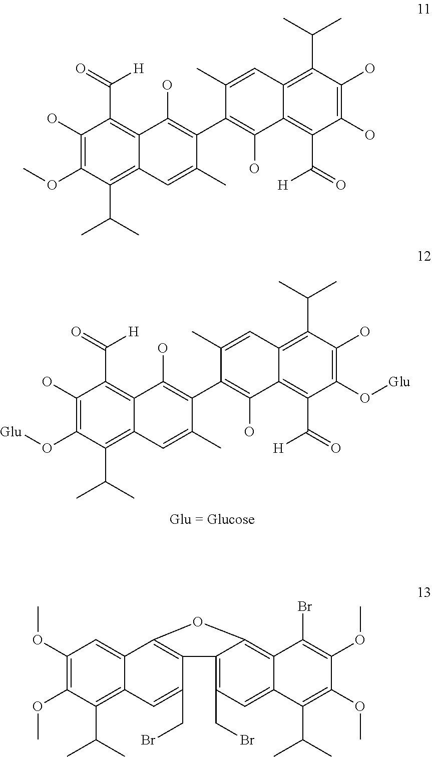

Furthermore, a feasibility of producing asymmetric gossypol derivatives based on tautomeric forms of gossypol has also been reported (Curr. Sci., 1973, 42, 821-822; Proceedings of the Alfred Benzon Symposium 26: Copenhagen, 1988, 75-100).

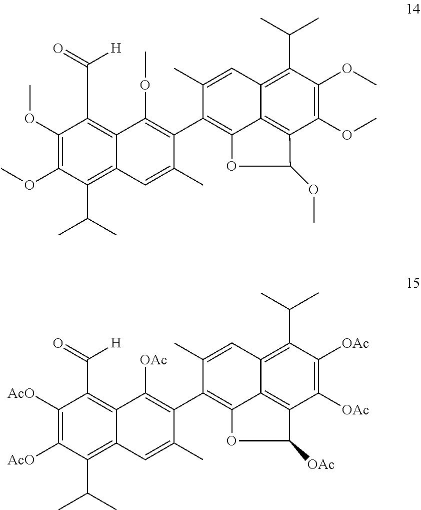

The most related to the present invention are the previously reported (Yao. Xue. Xue. Bao., 1987, Aug. 22, 8, 597-602) aldehyde derivatives (16) produced through a multistep synthesis including the Gattermann-Adams formylation. The same publication also suggests a feasibility of producing gossypol monoaldehyde (17), through a reaction in an aqueous solution of alkali in the presence of sodium hydrosulfite.

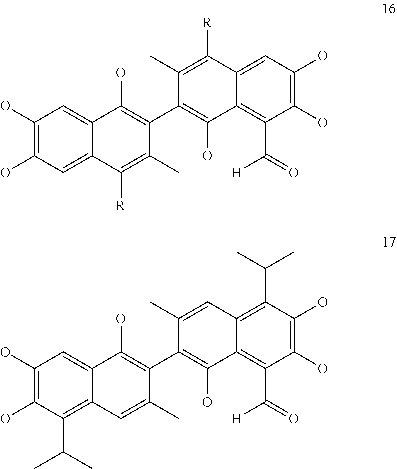

Thus, a necessity remains for developing novel asymmetric derivatives of the dinaphthalene series and processes for their labor-efficient production.

### SUMMARY OF THE INVENTION

The goal of the present invention is development of novel asymmetric derivatives of dinaphthalene polyphenols and processes for their production.

1) The present invention relates to novel asymmetric derivatives of dinaphthalene polyphenols of the general formula I:

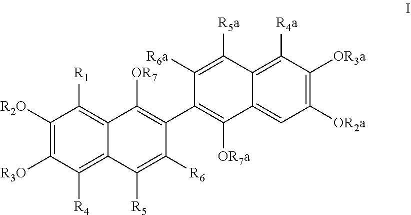

or general formula II:

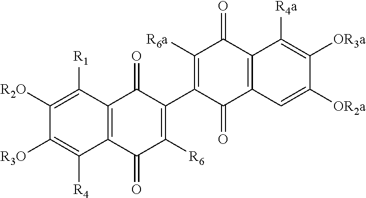

their tautomeric forms, pharmaceutically acceptable salts and solvates, wherein:

R1 represents: C(O)H, C═N—R8, C═N—NH—R8, C═N—NH—C(O)R8;

R8 represents: H; linear and branched C1-C20 alkyls, containing 0 to 10 substituents; linear and branched C2-C20 alkenyls and C2-C20 alkynyls with 1 to 9 double and (or) triple bonds, containing 0 to 10 substituents; unsubstituted and substituted cyclic systems with the number of atoms in the cycle of 3 to 20, containing 0 to 10 substituents, 0 to 10 double and (or) triple bonds, and 0 to 10 heteroatoms (N, S, O); unsubstituted and substituted aryls with 0 to 5 substituents; unsubstituted and substituted heteroaromatic compounds with the number of atoms in the cycle of 3 to 12 and the number of heteroatoms (N, S, O) of 1 to 6, containing 0 to 10 substituents; saturated, unsaturated and (or) aromatic polycyclic systems, containing 2 to 5 cycles with 3 to 8 atoms in the cycle, 0 to 10 double and (or) triple bonds and 0 to 10 heteroatoms (N, S, O), and 0 to 20 substituents;

R2, R3, R2a, R7, R7a independently represent R8 or C(O)R8;

R5 and R5a independently represent H or N═N—R9;

R9 represents: unsaturated and saturated aryls with 0 to 5 substituents; unsaturated and saturated heteroaromatic compounds with the number of atoms in the cycle of 3 to 12 and the number of heteroatoms (N, S, O) of 1 to 6, containing 0 to 10 substituents; saturated, unsaturated and (or) aromatic polycyclic systems, containing 2 to 5 cycles with the number of atoms in the cycle of 3 to 8 and the number of heteroatoms of 0 to 10, and 0 to 20 substituents;

R4, R4a, R6 and R6a independently represent H, linear and branched C1-C10 alkyls;

substituents represent H, F, Cl, Br, I, NO2, CN, SO3H, SO3M, SO2Cl, CF3, CCl3, CBr3, CI3, C(O)H, COOH, COOM, C(O)Cl, C(O)Br, C(O)I, R8, COOR8, C(O)R8, OH, OR8, OC(O)R8, OC(O)NR8R10, OSO2R8, NH2, NR8R10, N+(R8R10R11), NR8C(O)R10, NR8SO2R10, NR8C(O)NR10R11, SR8, S(O)R8, SO2R8, CH═NR8;

R10, R11 independently represent: H; linear and branched C1-C20 alkyls, containing 0 to 10 substituents; linear and branched C2-C20 alkenyls and C2-C20 alkynyls with 1 to 9 double and (or) triple bonds, containing 0 to 10 substituents; unsaturated and saturated cyclic systems with the number of atoms in the cycle of 3 to 20, containing 0 to 10 substituents, with 0 to 10 double and (or) triple bonds and the number of heteroatoms (N, S, O) of 0 to 10; unsaturated and saturated aryls with 0 to 5 substituents, unsaturated and saturated heteroaromatic compounds with the number of atoms in the cycle of 3 to 12 and the number of heteroatoms (N, S, O) of 1 to 6, containing 0 to 10 substituents; saturated, unsaturated and (or) aromatic polycyclic systems, containing 2 to 5 cycles with the number of atoms in the cycle of 3 to 8, the number of double and (or) triple bonds of 0 to 10, and the number of heteroatoms (N, S, O) of 0 to 10, and 0 to 20 substituents;

M represents Li, Na, K, Cs, Rb.

2) The present invention further relates to the compounds, structures of which are listed in Table 1.

TABLE 1

| | |
|---|---|
| I | 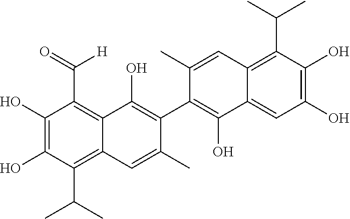 [SMILES: Cc1cc2c(C(C)C)c(O)c(O)cc2c(O)c1-c1c(C)cc2c(C(C)C)c(O)c(O)c(C=O)c2c1O] |
| II | 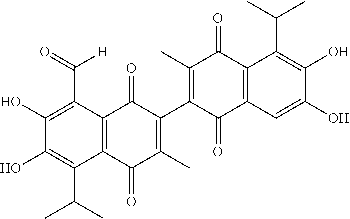 [SMILES: CC1=C(C2=C(C)C(=O)c3c(c(C=O)c(O)c(O)c3C(C)C)C2=O)C(=O)c2cc(O)c(O)c(C(C)C)c2C1=O] |
| III | 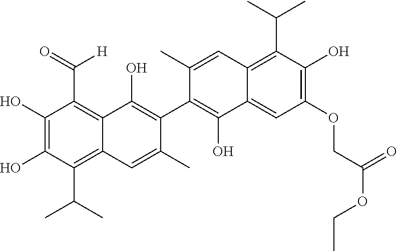 [SMILES: CCOC(=O)COc1cc2c(O)c(-c3c(C)cc4c(C(C)C)c(O)c(O)c(C=O)c4c3O)c(C)cc2c(C(C)C)c1O] |
| IV | 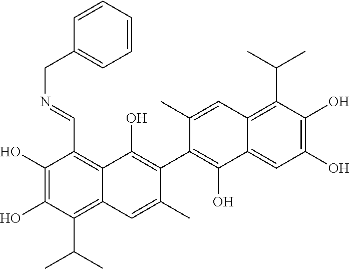 [SMILES: Cc1cc2c(C(C)C)c(O)c(O)cc2c(O)c1-c1c(C)cc2c(C(C)C)c(O)c(O)c(/C=N/Cc3ccccc3)c2c1O] |
| V | 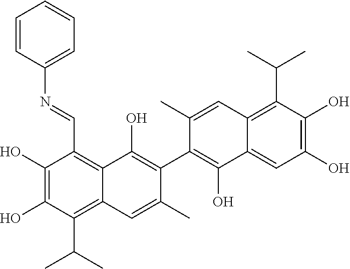 [SMILES: Cc1cc2c(C(C)C)c(O)c(O)cc2c(O)c1-c1c(C)cc2c(C(C)C)c(O)c(O)c(/C=N/c3ccccc3)c2c1O] |
| VI | 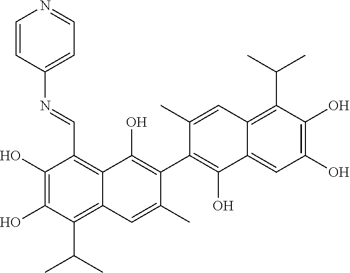 [SMILES: Cc1cc2c(C(C)C)c(O)c(O)cc2c(O)c1-c1c(C)cc2c(C(C)C)c(O)c(O)c(/C=N/c3ccncc3)c2c1O] |
| VII | 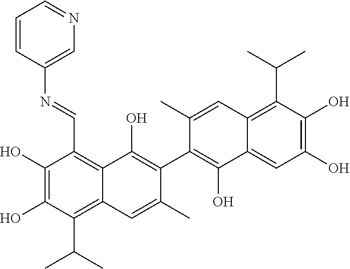 [SMILES: Cc1cc2c(C(C)C)c(O)c(O)cc2c(O)c1-c1c(C)cc2c(C(C)C)c(O)c(O)c(/C=N/c3cccnc3)c2c1O] |
| VIII | 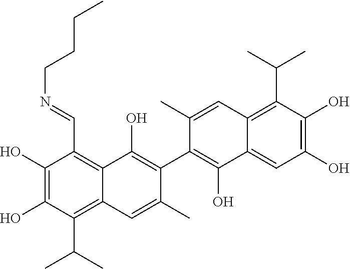 [SMILES: CCCC/N=C/c1c(O)c(O)c(C(C)C)c2cc(C)c(-c3c(C)cc4c(C(C)C)c(O)c(O)cc4c3O)c(O)c12] |
| IX | 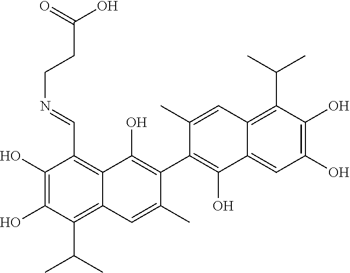 [SMILES: Cc1cc2c(C(C)C)c(O)c(O)cc2c(O)c1-c1c(C)cc2c(C(C)C)c(O)c(O)c(/C=N/CCC(=O)O)c2c1O] |
| X | 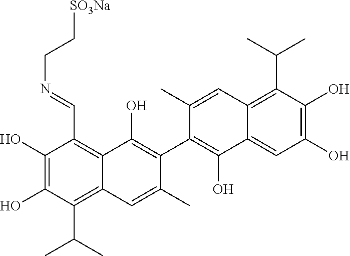 |
| XI | 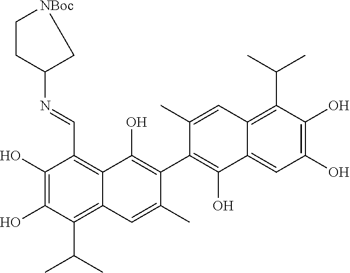 [SMILES: Cc1cc2c(C(C)C)c(O)c(O)cc2c(O)c1-c1c(C)cc2c(C(C)C)c(O)c(O)c(/C=N/C3CCN(C(=O)OC(C)(C)C)C3)c2c1O] |
| XII | 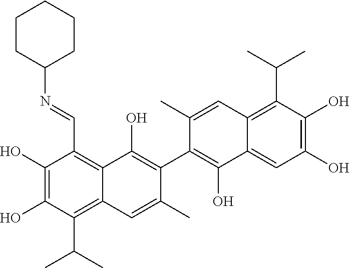 [SMILES: Cc1cc2c(C(C)C)c(O)c(O)cc2c(O)c1-c1c(C)cc2c(C(C)C)c(O)c(O)c(/C=N/C3CCCCC3)c2c1O] |
| XIII | 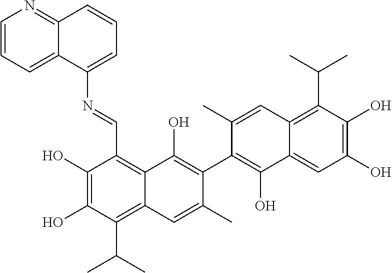 [SMILES: Cc1cc2c(C(C)C)c(O)c(O)cc2c(O)c1-c1c(C)cc2c(C(C)C)c(O)c(O)c(/C=N/c3cccc4ncccc34)c2c1O] |
| XIV | 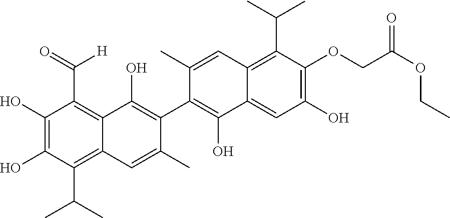 [SMILES: CCOC(=O)COc1c(O)cc2c(O)c(-c3c(C)cc4c(C(C)C)c(O)c(O)c(C=O)c4c3O)c(C)cc2c1C(C)C] |
| XV | 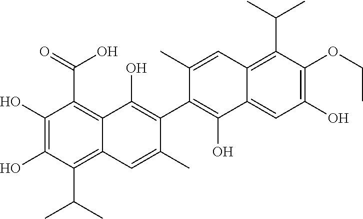 [SMILES: CCOc1c(O)cc2c(O)c(-c3c(C)cc4c(C(C)C)c(O)c(O)c(C(=O)O)c4c3O)c(C)cc2c1C(C)C] |
| XVI | 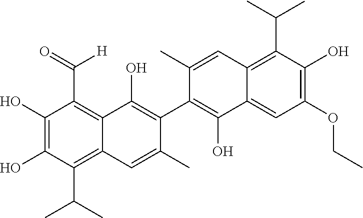 [SMILES: CCOc1cc2c(O)c(-c3c(C)cc4c(C(C)C)c(O)c(O)c(C=O)c4c3O)c(C)cc2c(C(C)C)c1O] |
| XVII | 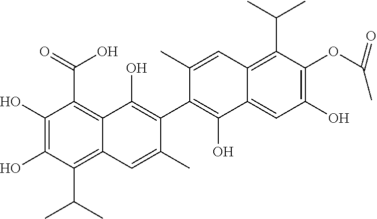 [SMILES: CC(=O)Oc1c(O)cc2c(O)c(-c3c(C)cc4c(C(C)C)c(O)c(O)c(C(=O)O)c4c3O)c(C)cc2c1C(C)C] |
| XVIII | 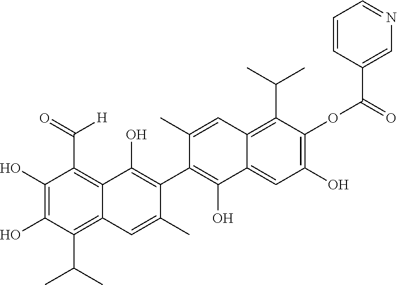 [SMILES: Cc1cc2c(C(C)C)c(OC(=O)c3cccnc3)c(O)cc2c(O)c1-c1c(C)cc2c(C(C)C)c(O)c(O)c(C=O)c2c1O] |
| XIX | 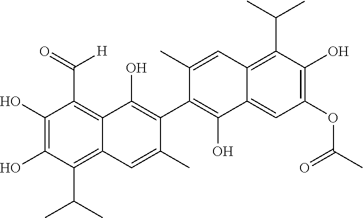 [SMILES: CC(=O)Oc1cc2c(O)c(-c3c(C)cc4c(C(C)C)c(O)c(O)c(C=O)c4c3O)c(C)cc2c(C(C)C)c1O] |
| XX | 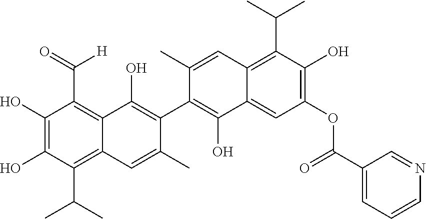 [SMILES: Cc1cc2c(C(C)C)c(O)c(OC(=O)c3cccnc3)cc2c(O)c1-c1c(C)cc2c(C(C)C)c(O)c(O)c(C=O)c2c1O] |
| XXI | 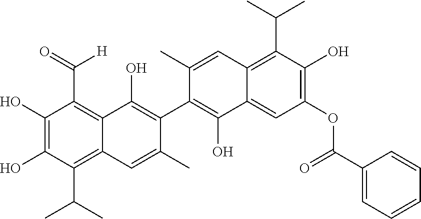 [SMILES: Cc1cc2c(C(C)C)c(O)c(OC(=O)c3ccccc3)cc2c(O)c1-c1c(C)cc2c(C(C)C)c(O)c(O)c(C=O)c2c1O] |
| XXII | 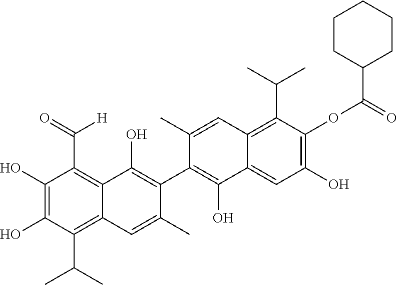 [SMILES: Cc1cc2c(C(C)C)c(OC(=O)C3CCCCC3)c(O)cc2c(O)c1-c1c(C)cc2c(C(C)C)c(O)c(O)c(C=O)c2c1O] |
| XXIII | 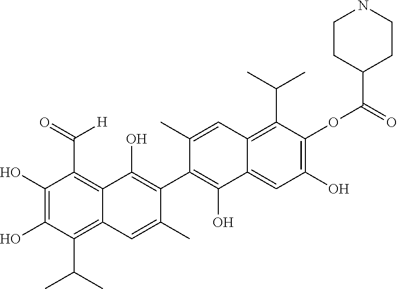 [SMILES: Cc1cc2c(C(C)C)c(OC(=O)C3CCNCC3)c(O)cc2c(O)c1-c1c(C)cc2c(C(C)C)c(O)c(O)c(C=O)c2c1O] |
| XXIV | 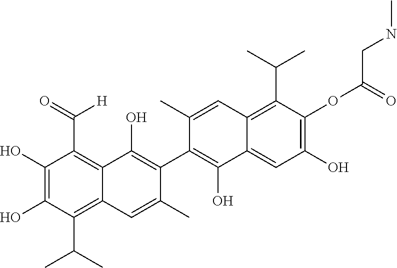 [SMILES: CNCC(=O)Oc1c(O)cc2c(O)c(-c3c(C)cc4c(C(C)C)c(O)c(O)c(C=O)c4c3O)c(C)cc2c1C(C)C] |
| XXV | 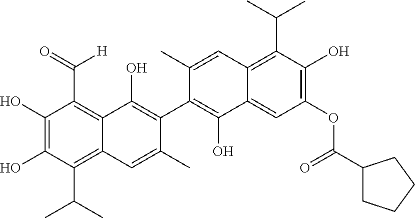 [SMILES: Cc1cc2c(C(C)C)c(O)c(OC(=O)C3CCCC3)cc2c(O)c1-c1c(C)cc2c(C(C)C)c(O)c(O)c(C=O)c2c1O] |
| XXVI | 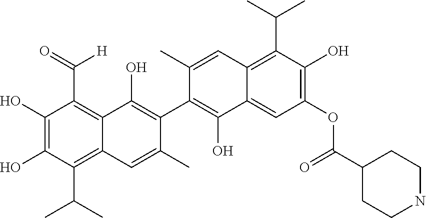 [SMILES: Cc1cc2c(C(C)C)c(O)c(OC(=O)C3CCNCC3)cc2c(O)c1-c1c(C)cc2c(C(C)C)c(O)c(O)c(C=O)c2c1O] |
| XXVII | 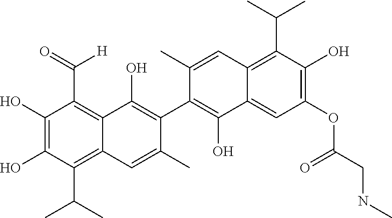 [SMILES: CNCC(=O)Oc1cc2c(O)c(-c3c(C)cc4c(C(C)C)c(O)c(O)c(C=O)c4c3O)c(C)cc2c(C(C)C)c1O] |
| XXVIII | 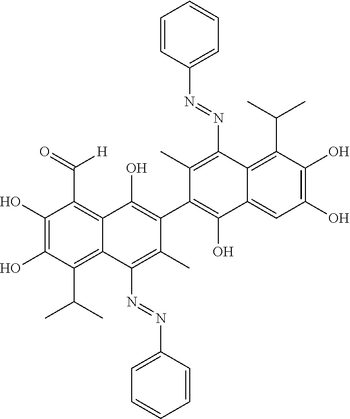 [SMILES: Cc1c(-c2c(C)c(/N=N/c3ccccc3)c3c(C(C)C)c(O)c(O)c(C=O)c3c2O)c(O)c2cc(O)c(O)c(C(C)C)c2c1/N=N/c1ccccc1] |
| XXIX | 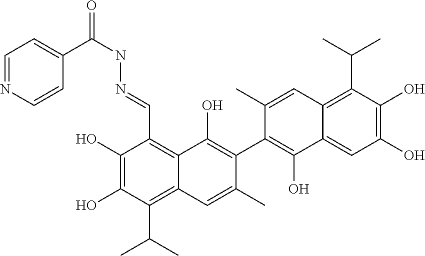 [SMILES: Cc1cc2c(C(C)C)c(O)c(O)cc2c(O)c1-c1c(C)cc2c(C(C)C)c(O)c(O)c(/C=N/NC(=O)c3ccncc3)c2c1O] |
| XXX | 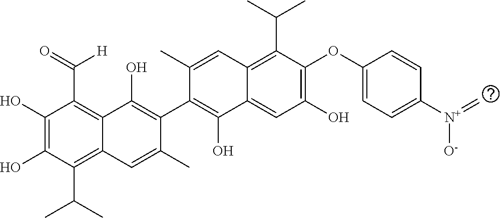 [SMILES: C=[N+]([O-])c1ccc(Oc2c(O)cc3c(O)c(-c4c(C)cc5c(C(C)C)c(O)c(O)c(C=O)c5c4O)c(C)cc3c2C(C)C)cc1] |
| XXXI | 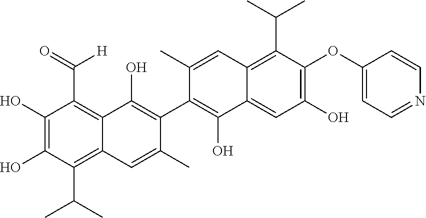 [SMILES: Cc1cc2c(C(C)C)c(Oc3ccncc3)c(O)cc2c(O)c1-c1c(C)cc2c(C(C)C)c(O)c(O)c(C=O)c2c1O] |
| XXXII | 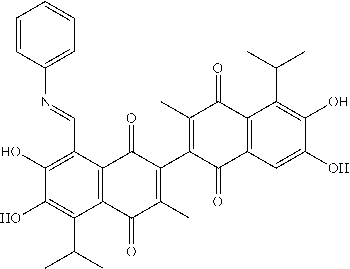 [SMILES: CC1=C(C2=C(C)C(=O)c3c(c(/C=N/c4ccccc4)c(O)c(O)c3C(C)C)C2=O)C(=O)c2cc(O)c(O)c(C(C)C)c2C1=O] |
| XXXIII | 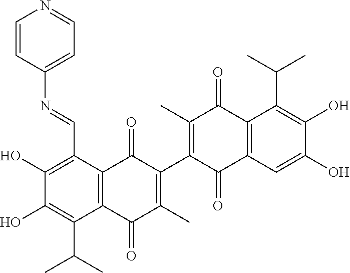 [SMILES: CC1=C(C2=C(C)C(=O)c3c(c(/C=N/c4ccncc4)c(O)c(O)c3C(C)C)C2=O)C(=O)c2cc(O)c(O)c(C(C)C)c2C1=O] |
| XXXIV | 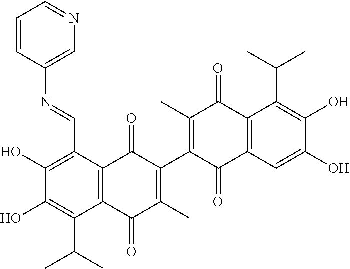 [SMILES: CC1=C(C2=C(C)C(=O)c3c(c(/C=N/c4cccnc4)c(O)c(O)c3C(C)C)C2=O)C(=O)c2cc(O)c(O)c(C(C)C)c2C1=O] |
| XXXV | 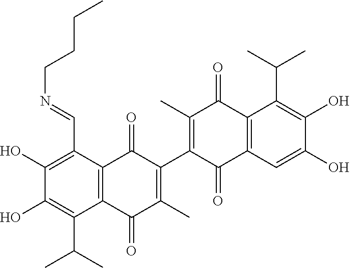 [SMILES: CCCC/N=C/c1c(O)c(O)c(C(C)C)c2c1C(=O)C(C1=C(C)C(=O)c3c(cc(O)c(O)c3C(C)C)C1=O)=C(C)C2=O] |
| XXXVI | 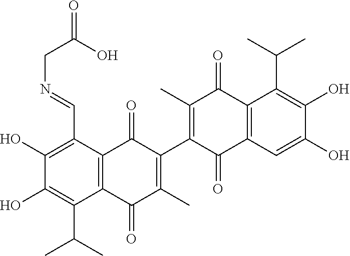 [SMILES: CC1=C(C2=C(C)C(=O)c3c(c(/C=N/CC(=O)O)c(O)c(O)c3C(C)C)C2=O)C(=O)c2cc(O)c(O)c(C(C)C)c2C1=O] |
| XXXVII | 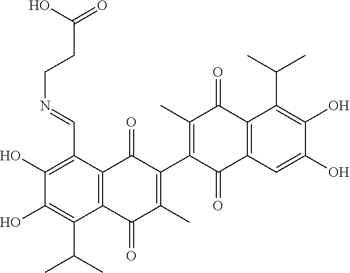 [SMILES: CC1=C(C2=C(C)C(=O)c3c(c(/C=N/CCC(=O)O)c(O)c(O)c3C(C)C)C2=O)C(=O)c2cc(O)c(O)c(C(C)C)c2C1=O] |
| XXXVIII | 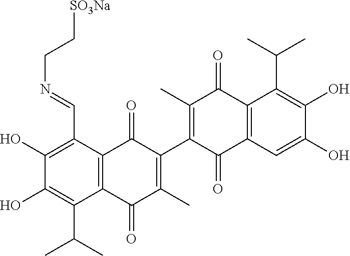 |
| XXXIX | 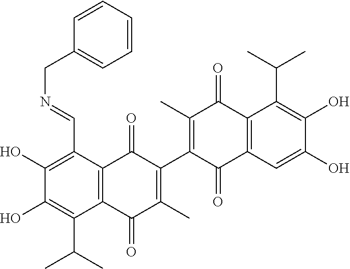 [SMILES: CC1=C(C2=C(C)C(=O)c3c(c(/C=N/Cc4ccccc4)c(O)c(O)c3C(C)C)C2=O)C(=O)c2cc(O)c(O)c(C(C)C)c2C1=O] |
| XL | 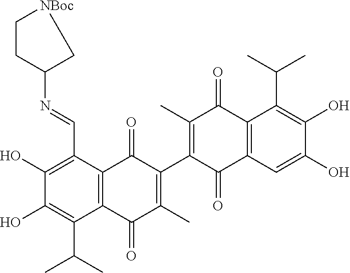 [SMILES: CC1=C(C2=C(C)C(=O)c3c(c(/C=N/C4CCN(C(=O)OC(C)(C)C)C4)c(O)c(O)c3C(C)C)C2=O)C(=O)c2cc(O)c(O)c(C(C)C)c2C1=O] |
| XL1 | 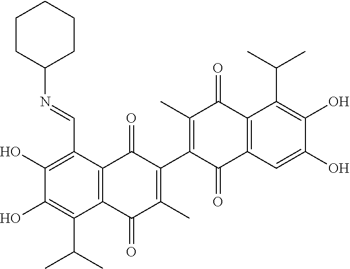 [SMILES: CC1=C(C2=C(C)C(=O)c3c(c(/C=N/C4CCCCC4)c(O)c(O)c3C(C)C)C2=O)C(=O)c2cc(O)c(O)c(C(C)C)c2C1=O] |
| XLII | 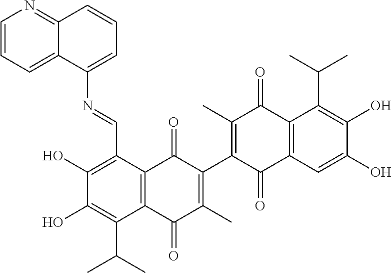 [SMILES: CC1=C(C2=C(C)C(=O)c3c(c(/C=N/c4cccc5ncccc45)c(O)c(O)c3C(C)C)C2=O)C(=O)c2cc(O)c(O)c(C(C)C)c2C1=O] |
| XLIII | 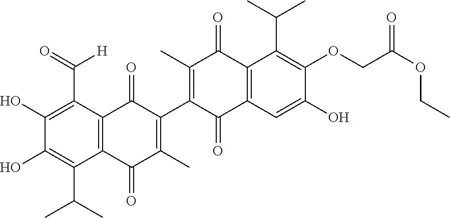 [SMILES: CCOC(=O)COc1c(O)cc2c(c1C(C)C)C(=O)C(C)=C(C1=C(C)C(=O)c3c(c(C=O)c(O)c(O)c3C(C)C)C1=O)C2=O] |
| XLIV | 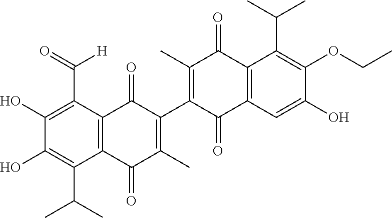 [SMILES: CCOc1c(O)cc2c(c1C(C)C)C(=O)C(C)=C(C1=C(C)C(=O)c3c(c(C=O)c(O)c(O)c3C(C)C)C1=O)C2=O] |
| XLV | 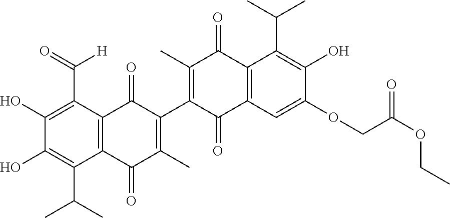 [SMILES: CCOC(=O)COc1cc2c(c(C(C)C)c1O)C(=O)C(C)=C(C1=C(C)C(=O)c3c(c(C=O)c(O)c(O)c3C(C)C)C1=O)C2=O] |
| XLVI | 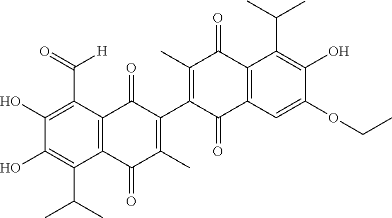 [SMILES: CCOc1cc2c(c(C(C)C)c1O)C(=O)C(C)=C(C1=C(C)C(=O)c3c(c(C=O)c(O)c(O)c3C(C)C)C1=O)C2=O] |
| XLVII | 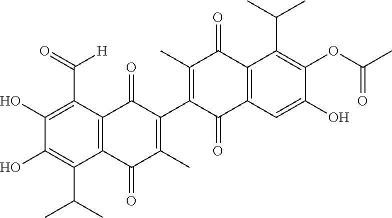 [SMILES: CC(=O)Oc1c(O)cc2c(c1C(C)C)C(=O)C(C)=C(C1=C(C)C(=O)c3c(c(C=O)c(O)c(O)c3C(C)C)C1=O)C2=O] |
| XLVIII | 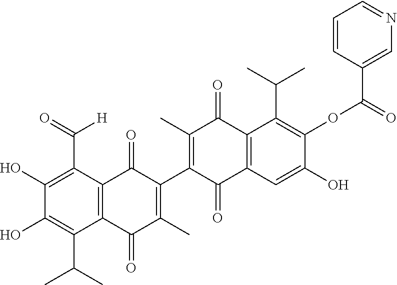 [SMILES: CC1=C(C2=C(C)C(=O)c3c(c(C=O)c(O)c(O)c3C(C)C)C2=O)C(=O)c2cc(O)c(OC(=O)c3cccnc3)c(C(C)C)c2C1=O] |
| XLIX | 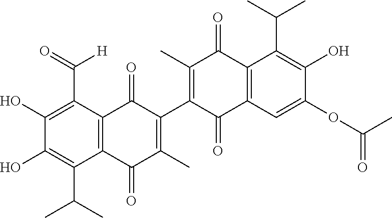 [SMILES: CC(=O)Oc1cc2c(c(C(C)C)c1O)C(=O)C(C)=C(C1=C(C)C(=O)c3c(c(C=O)c(O)c(O)c3C(C)C)C1=O)C2=O] |
| L | 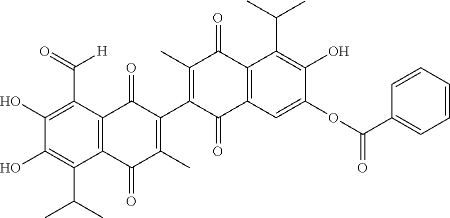 [SMILES: CC1=C(C2=C(C)C(=O)c3c(c(C=O)c(O)c(O)c3C(C)C)C2=O)C(=O)c2cc(OC(=O)c3ccccc3)c(O)c(C(C)C)c2C1=O] |
| LI | 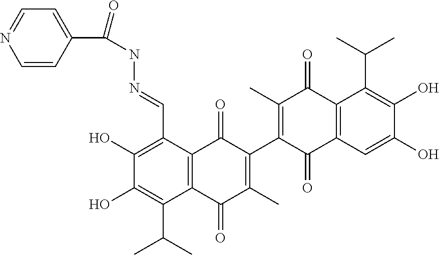 [SMILES: CC1=C(C2=C(C)C(=O)c3c(c(/C=N/NC(=O)c4ccncc4)c(O)c(O)c3C(C)C)C2=O)C(=O)c2cc(O)c(O)c(C(C)C)c2C1=O] |
| LII | 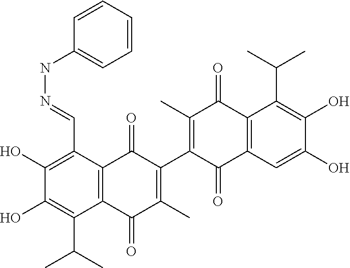 [SMILES: CC1=C(C2=C(C)C(=O)c3c(c(/C=N/Nc4ccccc4)c(O)c(O)c3C(C)C)C2=O)C(=O)c2cc(O)c(O)c(C(C)C)c2C1=O] |
| LIII | 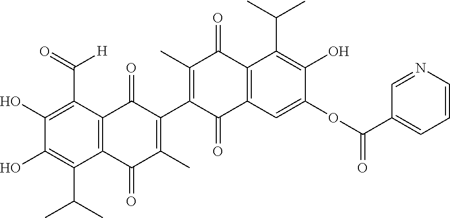 [SMILES: CC1=C(C2=C(C)C(=O)c3c(c(C=O)c(O)c(O)c3C(C)C)C2=O)C(=O)c2cc(OC(=O)c3cccnc3)c(O)c(C(C)C)c2C1=O] |
| LIV | 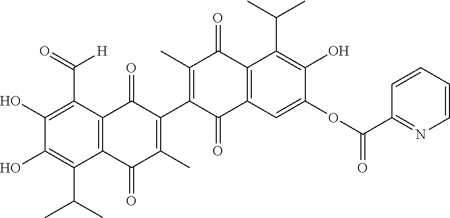 [SMILES: CC1=C(C2=C(C)C(=O)c3c(c(C=O)c(O)c(O)c3C(C)C)C2=O)C(=O)c2cc(OC(=O)c3ccccn3)c(O)c(C(C)C)c2C1=O] |
| LV | 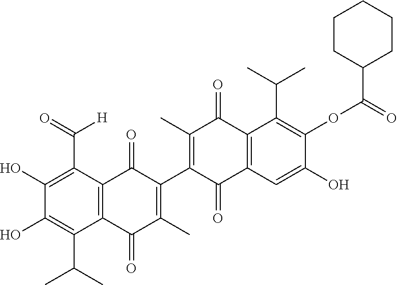 [SMILES: CC1=C(C2=C(C)C(=O)c3c(c(C=O)c(O)c(O)c3C(C)C)C2=O)C(=O)c2cc(O)c(OC(=O)C3CCCCC3)c(C(C)C)c2C1=O] |
| LVI | 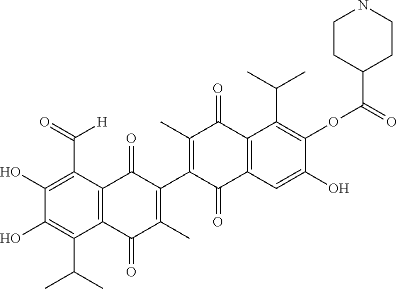 [SMILES: CC1=C(C2=C(C)C(=O)c3c(c(C=O)c(O)c(O)c3C(C)C)C2=O)C(=O)c2cc(O)c(OC(=O)C3CCNCC3)c(C(C)C)c2C1=O] |
| LVII | 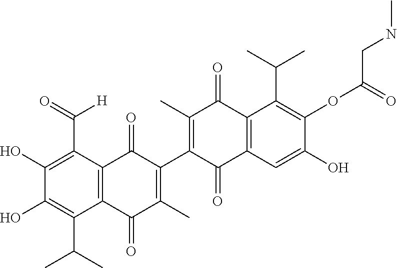 [SMILES: CNCC(=O)Oc1c(O)cc2c(c1C(C)C)C(=O)C(C)=C(C1=C(C)C(=O)c3c(c(C=O)c(O)c(O)c3C(C)C)C1=O)C2=O] |
| LVIII | 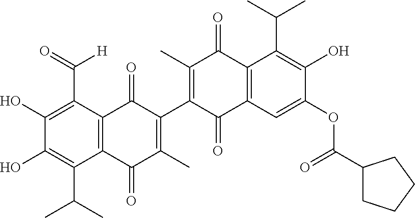 [SMILES: CC1=C(C2=C(C)C(=O)c3c(c(C=O)c(O)c(O)c3C(C)C)C2=O)C(=O)c2cc(OC(=O)C3CCCC3)c(O)c(C(C)C)c2C1=O] |
| LIX | 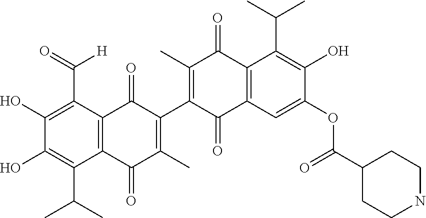 [SMILES: CC1=C(C2=C(C)C(=O)c3c(c(C=O)c(O)c(O)c3C(C)C)C2=O)C(=O)c2cc(OC(=O)C3CCNCC3)c(O)c(C(C)C)c2C1=O] |
| LX | 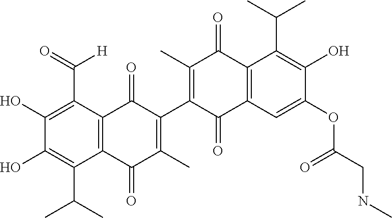 [SMILES: CNCC(=O)Oc1cc2c(c(C(C)C)c1O)C(=O)C(C)=C(C1=C(C)C(=O)c3c(c(C=O)c(O)c(O)c3C(C)C)C1=O)C2=O] |
| LXI | 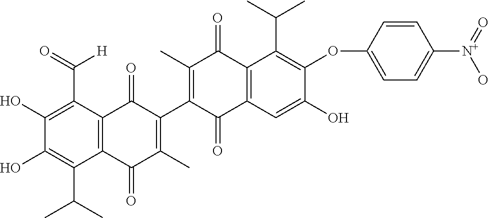 |
| LXII | 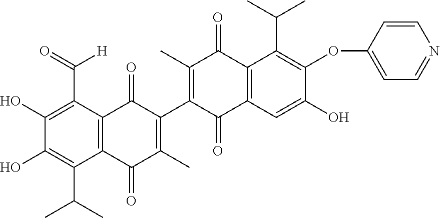 [SMILES: CC1=C(C2=C(C)C(=O)c3c(c(C=O)c(O)c(O)c3C(C)C)C2=O)C(=O)c2cc(O)c(Oc3ccncc3)c(C(C)C)c2C1=O] |

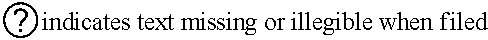

3) The present invention further relates to the process for producing compounds of the general formula (I) according to the item (1), including the following stages:

treatment of the gossypol derivative, not necessarily in its solvate form, with an aqueous solution of alkali or alkaline earth metal hydroxide;

treatment of the resultant reaction mixture with organic and/or inorganic acids to pH=0.1-10, preferably 3-5;

the reaction product isolation and purification;

and, if necessary, subsequent treatment of resultant product with a reagent, which is selected from a group containing: aliphatic, aromatic or heteroaromatic amines; alkylating agents, acylating agents; diazonium salts;and

target product isolation and purification.

Preferably, the gossypol derivative represents gossypol, gossypol acetic acid, methyl ethers of gossypol.

Preferably, the gossypol derivative is treated with an aqueous solution of sodium or potassium hydroxide.

Preferably, the concentration of the aqueous solution of alkali or alkaline earth metal hydroxide is 0.1-50 mass %, preferably 10-30 mass %.

Preferably, the treatment with aliphatic, aromatic or heteroaromatic amines is performed in a solvent, such as isopropanol, diethyl ether, dioxane or tetrahydrofuran.

Preferably, aromatic or aliphatic halogen derivatives or alkyl sulfates are used as alkylating agents, and treatment is performed in the presence of organic and/or inorganic bases, such as cesium carbonate, sodium ethylate, potassium tert-butylate, triethylamine or sodium hydride, in a solvent, such as isopropanol, diethyl ether or tetrahydrofuran.

Preferably, carbonic acids or halogen anhydrides are used as an acylating agent, and treatment is performed in the presence of organic and/or inorganic bases, such as potassium carbonate, cesium carbonate, sodium methylate, sodium ethylate, potassium tert-butylate, triethylamine, diisopropylamine or sodium hydride, not necessarily in the presence of coupling reagents such as carbonyldiimidazol, DCC, in a solvent such as isopropanol, diethyl ether or tetrahydrofuran.

Preferably, treatment with diazonium salts is performed in the aqueous environment and/or in a solvent, such as ethanol or diethyl ether.

4) The present invention further relates to the process for producing compounds of the general formula (II) according to the item (1), including the following stages:

treatment of gossypol derivative, not necessarily in the form of its solvate, with an aqueous solution of alkali and/or alkaline earth metal hydroxide;

treatment of the reaction mixture with organic and/or inorganic acids to pH=0.1-10, preferably 3-5;

the resultant product isolation and purification;

subsequent treatment of the resultant product with hydrogen peroxide at the concentration of 0.1-99.8% in organic and/or inorganic acids; or

subsequent treatment of the resultant product with trivalent ferric compounds in the presence of an organic solvent and/or an organic or inorganic acid;

the resultant product isolation and purification; and

if necessary, subsequent treatment of resultant product with a reagent selected from a group containing: aliphatic, aromatic or heteroaromatic amines; alkylating agents and acylating agents; and

target product isolation and purification.

Preferably, gossypol derivative represents gossypol, gossypol acetic acid, gossypol methyl ether.

Preferably, gossypol derivative is treated with an aqueous solution of potassium hydroxide or sodium hydroxide.

Preferably, the concentration of the aqueous solution of alkali or alkaline earth metal hydroxide is 0.1-50 mass %, preferably 10-30 mass %.

Preferably, hydrogen peroxide is used at the concentration of 1-30 mass % and treatment is performed in the acetic acid environment.

Preferably, trivalent ferric compound represents ferric chloride (III) in the solid form or in an aqueous solution, and treatment is performed in the acetone and/or acetic acid environment.

Preferably, treatment with aliphatic, aromatic or heteroaromatic amines is performed in a solvent, such as isopropanol, diethyl ether, dioxane or tetrahydrofuran.

Preferably, aromatic or aliphatic halogen derivatives or alkyl sulfates are used as alkylating agents, and treatment is performed in the presence od organic and/or inorganic bases, such as cesium carbonate, sodium ethylate, potassium tert-butylate, triethylamine or sodium hydride, in a solvent, such as isopropanol, diethyl ether or tetrahydrofuran.

Preferably, carbonic acids or halogen anhydrides are used as an acylating agent, and treatment is performed in the presence of organic and/or inorganic bases, such as potassium carbonate, cesium carbonate, sodium methylate, sodium ethylate, potassium tert-butylate, triethylamine, diisopropylamine or sodium hydride, not necessarily in the presence of coupling reagents such as carbonyldiimidazol, DCC, in a solvent such as chloroform, tetrahydrofuran or dioxane.

5) The present invention further relates to an antiviral agent, which represents a compound of the general formula (I) or (II) according to the item (1) or compounds according to the item (2).

In the preferable case, the present invention relates to an antiviral agent effective against influenza, herpes, hepatitis, HIV viruses.

### FIGURE LEGENDS

FIG. 1 shows chromatograms of the compounds of formulas I and II.

FIG. 2 shows mass-spectra of the compounds of formulas I and II.

FIG. 3 shows UV-spectra of the compounds of formulas I and II.

FIG. 4 shows chromatograms of (+) and (−) isomers of the compound of formula I.

FIG. 5 shows IR-spectra of gossypol and the compound of formula I in a tablet of potassium bromide.

### DETAILED DESCRIPTION OF THE INVENTION

In the context of the present description:

the term “C1-C20 alkyl” means a linear or branched hydrocarbon chain, containing 1-20 atoms of carbon, preferably 1-10 atoms of carbon, containing 0-10 substituents, preferably 1-3 substituents, particularly methyl, ethyl, n-propyl, isopropyl, n-butyl, isobutyl, C2H4COOH, 3-methoxybenzyl;

the terms “C1-C20 alkenyl” and “C2-C20 alkynyl” mean a linear or branched hydrocarbon chain, containing 2-20 atoms of carbon, preferably 2-7 atoms of carbon, with 1-9 double and (or) triple bonds, preferably 1-3 double and (or) triple bonds, containing 0-10 substituents, preferably 0-3 substituents, particularly CH2CH═CHC2H4OCH3, CH2CH═CHC6H5, CH2C≡CH, CH2C≡CC6H5;

the term “cyclic system” means a non-aromatic monocyclic structure with the number of atoms in the cycle of 3-20, preferably 5-7 atoms, containing 0-10 substituents, preferably 0-3 substituents, with 0-10 double and (or) triple bonds, preferably 0-2 bonds, and the number of heteroatoms (N, S, O) of 0 to 10, preferably 0-2 heteroatoms, particularly tetrahydropyran-4-yl, cycloheptyl, tetrahydrothiopyran-4-yl, pyrrolidin-2-on-3-yl, 1-ethylazepan-2-on-3-yl;

the term “aryl” means a six-member aromatic compound of the benzene series, containing 0-5 substituents, preferably 0-3 substituents, particularly, 4-carboxyphenyl, 3-hydroxy-4-metoxyphenyl, 3-methylphenyl;

the term “heteroaromatic compound” means a monocyclic aromatic structure with the number of atoms in the cycle of 3 to 12, preferably 5-7 atoms, the number of heteroatoms (N, S, O) of 1-6, preferably 0-2 heteroatoms, containing 0-10 substituents, preferably 0-3 substituents, particularly pyridazine-4-yl, pyrazin-2-yl, pyrimidin-5-yl;

the term “polycyclic system” means an aromatic and (or) non-aromatic condensed cyclic compound, containing 2-5 cycles, preferably 2-3 cycles, with the number of atoms in the cycle of 3-8, preferably 5-7 atoms, 0-10 double and (or) triple bonds, preferably 0-3 bonds, the number of heteroatoms (N, S, O) of 0-10, preferably 0-4 heteroatoms, and containing 0-20 substituents, preferably 0-3 substituents, particularly quinolin-6-yl, 1-acetylindolin-6-yl, imidazo[1,2-a]pyrimidin-3-yl;

the term “solvates” means the products of attachment of solvent or reagent to the dissolved substance, as well as clathrates—compounds including molecules of solvent or reagent in the crystal structure cavity of another substance, present in the solution and (or) solid state, particularly gossypol acetic acid, clathrate of gossypol with isobutyl acetate, clathrate of bis-o-toluidine gossypol with 1,4-dioxane;

the term “pharmaceutically acceptable salts” means substances that dissociate in aqueous solutions into cations and anions of acid residues, particularly chlorides, sulfates, acetates, carbonates, oxalates, carb oxyl ate s, alcoholates, phenolates, ammonium salts, pyridinium salts;

the term “alkali hydroxide” means the compound consisting of alkali metal and a hydroxyl group, particularly lithium hydroxide, sodium hydroxide, potassium hydroxide;

the term “alkaline earth hydroxide” means the compound consisting of alkaline earth metal and a hydroxyl group, particularly calcium hydroxide, magnesium hydroxide, barium hydroxide;

the term “organic acid” means an organic compound possessing acidic properties, particularly formic acid, acetic acid, oxalic acid, p-toluenesulfonic acid, citric acid, tartaric acid;

the term “inorganic acid” means an inorganic compound possessing acidic properties, particularly hydrochloric acid, sulphuric acid, orthophosphoric acid, perchloric acid, carbonic acid, nitric acid;

the term “aliphatic amine” means an organic compound that belongs to the class of amines and is differentiated by containing non-aromatic substituents at the nitrogen atom, particularly methylamine, morpholine, isopentylamine, benzylamine, glycine, 4-aminopiperidine;

the term “aromatic amine” means a derivative of aromatic hydrocarbons, in which one or more hydrogen atoms are replaced by the amino group, particularly 2-methyl-4-methoxyaniline, 3-fluoroaniline, 3-ethoxyaniline, 4-nitroaniline;

the term “heteroaromatic amine” means a derivative of heteroaromatic compounds, in which one or more hydrogen atoms are replaced by the amino group, particularly 4-aminopyrimidine, methyl 2-amino-6,7-dihydro-5H-pyrrolo[1,2-A]imidazole-3-carboxylate, 1-H-indole-3-amine, 5-aminoindole, 5-aminoindane, 6-amino-2,2-dimethyl-chroman-4-one;

the term “alkylating agent” means the substance that allows for introducing the alkyl group into a molecule, particularly benzyl bromide, dimethyl sulfate, methyl iodide, ethyl 4-bromobutyrate;

the term “acylating agent” means the substance that allows for introducing the acyl group into a molecule, particularly acetic anhydride, p-ethoxybenzoyl chloride, trifluoroacetic anhydride, cyclopentane carbonyl chloride, acetyl bromide;

the term “diazonium salt” means the compound of formula R—[N+≡N]X—, where R-aryl or hetaryl, X— a corresponding acid anion, particularly phenyl diazonium chloride, p-methoxyphenyl diazonium hydrosulfate;

the term “methyl ether of gossypol” means a gossypol derivative in any tautomeric form, in which one or more hydrogen atoms in the hydroxyl group are replaced by methyl, particularly 1,1′, 7,7′-tetrahydroxy-5,5′-diisopropyl-6,6′-dimethoxy-3,3′-dimethyl-2,2′-binaphthalene-8,8′-dicarbaldehyde, 6,6′, 7,7′-tetrahydroxy-5,5′-diisopropyl-1,1′-dimethoxy-3,3′-dimethyl-2,2′-binaphthalene-8,8′-dicarbaldehyde;

the term “aliphatic halogen derivative” means any non-aromatic compound, in which one and (or) several hydrogen atoms are replaced by halogen, particularly ethyl bromide, cyclopentyl chloride, 3,5-dimethoxybenzyl bromide;

the term “aromatic halogen derivative” means any aromatic and (or) heteroaromatic organic compound, in which one and (or) several hydrogen atoms are replaced by halogen, particularly 1-fluoro-4-nitrofluorobenzene, 2-chloropyridine, 2-bromo-1,3,4-thiodiazole;

the term “organic base” means any organic compound capable of accepting positively charged ions, particularly triethylamine, 4-methylmorpholine, N-ethyl diisopropylamine, potassium tert-butylate;

the term “inorganic base” means any inorganic compound capable of accepting positively charged ions, particularly sodium carbonate, potassium hydroxide, sodium acetate, sodium hydroxide, sodium bicarbonate, cesium carbonate, potassium carbonate;

the term “carbonic acid” means an organic compound containing in its structure at least one COOH group, particularly acetic acid, formic acid, citric acid, trifluoroacetic acid, benzoic acid;

the term “halogen anhydride” means an organic compound comprising a carbonic acid residue with its hydroxyl group replaced by halogen, particularly cyclopropane carbonyl chloride, 4,4,4-trifluorobutyryl bromide, 5-methylnicotinoyl chloride;

the term “coupling reagent” means the compound employed in the organic synthesis for the carboxyl group activation, particularly carbonyl diimidazole, 1-ethyl-3-(3-dimethylaminopropyl)carbodiimide, 1,3-dicyclohexylcarbodiimide;

The term “tautomeric forms” means substances of the same chemical composition, capable of spontaneous transformation into structural isomers-tautomers by means of an atomic group migration between several centers within the molecule, example shown below (A—aldehyde, B—ketone, C—hemiacetal, D—imine, F—enamine).

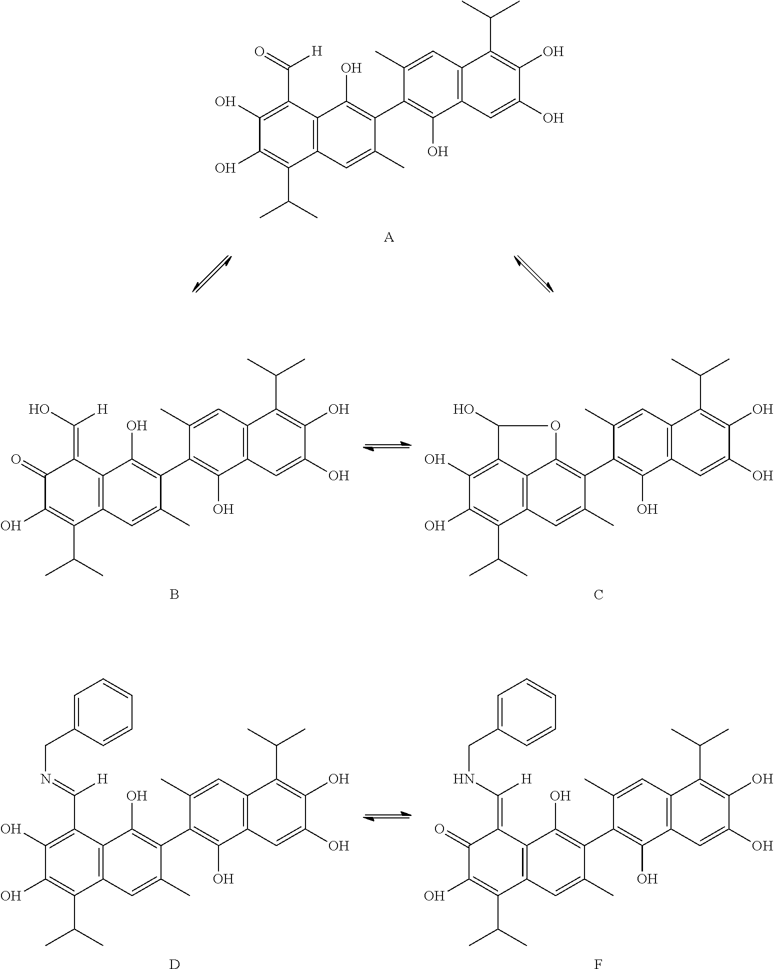

The present invention is illustrated by the following examples.

Synthesis of compound I (1,1′,6,6′,7,7′-hexahydroxy-5,5′-diisopropyl-3,3′-dimethyl-2,2′-binaphthalene-8-carbaldehyde).

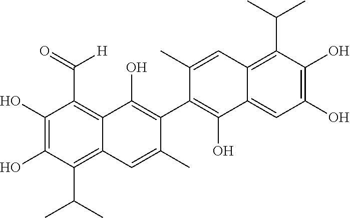 [SMILES: Cc1cc2c(C(C)C)c(O)c(O)cc2c(O)c1-c1c(C)cc2c(C(C)C)c(O)c(O)c(C=O)c2c1O]

Process 1: To a 3000 mL chemical beaker containing 1500 mL of 17% sodium hydroxide 70 g (0.12 mol) of gossypolacetic acid (GAA) is added at magnetic stirring. The reaction mixture is stirred at room temperature for 8 hours, after which kept for additional 15 hours without stirring. Then the reaction mixture is acidified with acetic acid to pH=4.0. The formed residue is filtered, washed two times with distilled water, and then dried in a dry heat oven at 50° C. to the constant weight. The resultant powder is purified by recrystallization or using chromatographic methods. The product represents yellow powder with the yield of 35 g (53%).

NMR 1H (CDCl3, ppm., J, Hz): 1.51-1.54 m (12H, 4CH3, 2 i-Pr), 2.10 s (3H, CH3), 2.13 s (3H, CH3), 3.85-3.90 m (2H, 2CH, 2 i-Pr), 5.24 s (1H, OH), 5.86 s (1H, OH), 5.91 s (1H, OH), 5.98 s (1H, OH), 6.39 s (1H, OH), 7.45 s (1H, CH), 7.62 s (1H, CH), 7.74 s (1H, CH), 11.12 s (1H, CHO), 15.15 (1H, OH); NMR 13C (CDCl3, δ, ppm.): 199.6 (C, HC═O), 156.1 (C, Ph-OH), 150.4 (C, Ph-OH), 149.4 (C, Ph-OH), 143.3 (C, Ph-OH), 142.7 (C, Ph-OH), 134.3 (C, Ph-OH), 134.2 (C, Ph-i-Pr), 132.8 (C, Ph-i-Pr), 130.3 (C, Ph-CH3), 129.4 (C, Ph-CH3), 129.1 (C, Ph), 128.3 (C, Ph), 125.9 (CH, Ph), 125.4 (CH, Ph), 117.8 (C, Ph-Ph), 116.9 (C, Ph-Ph), 114.5 (C, Ph), 111.9 (C, Ph), 110.8 (C, Ph-CH═O), 103.2 (CH, Ph), 27.2 (CH, i-Pr), 27.0 (CH, i-Pr), 20.0-20.9 (4+2, 4CH3,×2 i-Pr+2 CH3, ×2 Ph-CH3); ESI-MS m/z: 489.2 [M-H]—(C29H30O7, Mr=490.2); UV-VIS: λmax/nm: 249, 289, 379.

Synthesis of compound II (6,6′,7,7′-tetrahydroxy-5,5′-diisopropyl-3,3′-dimethyl-1,1′,4,4′-tetraoxo-1,1′,4,4′-tetrahydro-2,2′-binaphthalene-8-carbaldehyde).

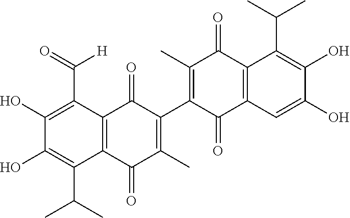 [SMILES: CC1=C(C2=C(C)C(=O)c3c(c(C=O)c(O)c(O)c3C(C)C)C2=O)C(=O)c2cc(O)c(O)c(C(C)C)c2C1=O]

The solution of 4 g (0.008 mol) of compound I in 100 mL of acetone and 200 mL of acetic acid is heated on a water bath, while 150 mL of 10% aqueous solution of ferric chloride hexahydrate III is added over several minutes. The solution is cooled and 250 mL of water is added. The formed dark residue, containing iron compounds, is removed by treatment with a mixture of ether and aqueous solution of 20% sulphuric acid. Purified phenol is extracted with ether and the ether layer is separated, dried over sodium sulfate, and evaporated to dryness. The obtained powder is purified by recrystallization or using chromatographic methods. The product represents orange powder with the yield of 3.1 g (69%).

To 1.7 g (0.0035 mol) of compound I in a 50 mL flat-bottomed flask, 17 mL of acetic acid and 2 mL of 30% hydrogen peroxide solution are added. The reaction mass is mixed at room temperature for 24 hours. After that, the reaction mixture is poured into 100 mL distilled water, extracted three times with 20 mL portion of chloroform, and the combined organic layer is washed two times with 30 mL portions of distilled water. The organic layer is separated, dried over sodium sulfate, and evaporated to dryness. The obtained powder is purified by recrystallization or using chromatographic methods. The product represents orange powder with the yield of 1.0 g (54%).

NMR 1H (CDCl3,ppm, J, Hz): 1.35-1.44 m (12H, 4CH3, 2 i-Pr), 2.02 s (3H, CH3), 2.03 s (3H, CH3), 4.09-4.14 m (1H, CH, 1 i-Pr), 4.28-4.33 m (1H, CH, 1 i-Pr), 6.23 s (1H, OH), 6.59 s (1H, OH), 6.93 c (1H, OH), 7.46 c (1H, CH), 10.53 c (1H, CHO), 12.93 (1H, OH); NMR 13C (DMSO-d6, CDCl3, δ, ppm.): 198.2 (C, HC═O), 186.9 (C, C═O), 186.8 (C, C═O), 184.3 (C, C═O), 182.7 (C, C═O), 153.0 (C, Ph-OH), 150.8 (C, Ph-OH), 150.7 (C, Ph-OH), 149.0 (C, Ph-OH), 147.9 (C, Ph-i-Pr), 146.4 C, Ph-i-Pr), 141.7 (C, Ph-CH3), 138.8 (C, Ph-CH3), 137.4 (C, Ph), 137.3 (C, Ph), 127.7 (C, Ph-Ph), 126.7 (C, Ph-Ph), 126.4 (C, Ph), 125.1 (C, Ph), 116.7 (C, Ph-CH═O), 111.3 (CH, Ph), 28.6 (CH, i-Pr), 27.3 (CH, i-Pr), 19.8-20.2 (4C, 4CH3, i-Pr), 15.1 (C, Ph-CH3), 14.7 (C, Ph-CH3); ESI-MS m/z: 517.2 [M-H]—(C29H26O9, Mr=518.2); UV-VIS: λmax/nm: 277, 368.

Syntesis of compound III (ethyl [(8-formyl-1,1′,6,6′,7′-pentahydroxy-5,5′-diisopropyl-3,3′-dimethyl-2,2′-binaphthalen-7-yl)oxy]acetate).

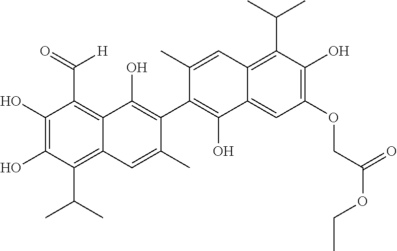 [SMILES: CCOC(=O)COc1cc2c(O)c(-c3c(C)cc4c(C(C)C)c(O)c(O)c(C=O)c4c3O)c(C)cc2c(C(C)C)c1O]

Ten mL of tetrahydrofuran, 1.00 g (2.0 mmol) of compound I and 0.50 g (4.0 mmol) of potassium tert-butylate are placed in a test tube, and the reaction mass is mixed for 20 minutes. Then 0.90 g (4.0 mmol) of ethyl iodoacetate is added, and the reaction mixture is mixed at heating for 8 hours. The reaction mixture is poured into 50 mL of water, acidified with acetic acid to pH 5, and extracted two times with 10 mL portions of chloroform. The combined organic layers are dried over sodium sulfate and concentrated under reduced pressure. The obtained powder is purified by recrystallization or using chromatographic methods. The product represents yellow powder with the yield of 0.3 g (27%).

NMR 1H (CDCl3, ppm, J, Hz): 1.29-1.32 t (2H, CH2COOCH2CH3, 2J=7.1), 1.54-1.58 m (12H, 4CH3, 2 i-Pr), 2.12 s (3H, CH3), 2.14 s (3H, 2CH3), 3.84-3.91 m (2H, 2CH, 2 i-Pr), 4.27-4.31 sq (2H, CH2COOCH2CH3, J=7.1), 4.79 s (2H, CH2COOCH2CH3), 5.17 s (1H, OH), 5.74 s (1H, OH), 6.41 s (1H, OH), 6.84 s (1H, OH), 7.45 s (1H, CH), 7.66 s (1H, CH), 7.75 s (1H, CH), 11.14 s (1H, CHO), 15.24 (1H, OH); ESI-MS m/z: 575.2 [M-H]—(C33H39O9, Mr=576.2).

Syntesis of compound IV (8-(benzylimino)methyl-5,5′-diisopropyl-3,3′-dimethyl-2,2′-binapthalene-1,1′,6,6′,7,7′-hexol).

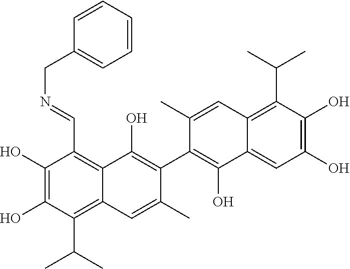 [SMILES: Cc1cc2c(C(C)C)c(O)c(O)cc2c(O)c1-c1c(C)cc2c(C(C)C)c(O)c(O)c(/C=N/Cc3ccccc3)c2c1O]

To 1 ml of diethyl ester in a test tube, 0.001 g (0.2 mmol) of compound I and then 0.022 g (0.2 mmol) of benzylamine are added. The reaction mass is mixed at room temperature for 2 hours. The formed residue is filtered and washed with diethyl ether. The resultant product represents yellow powder with the yield of 0.060 g (48%).

NMR 1H (DMSO-d6, ppm, J, Hz): 1.38-1.41 m (12H, 4CH3, 2 i-Pr), 1.90 s (3H, CH3), 1.92 s (3H, CH3), 3.67-3.79 m (2H, 2CH, 2 i-Pr), 4.71 s (2H, C6H5CH2N), 7.25-7.43 m (8H+3H, C6H5CH2N, CH), 7.45 s (1H, OH), 7.63 s (1H, OH), 7.88 s (1H, OH), 8.25 s (1H, OH), 8.33 s (1H, OH), 9.82-9.84 d (1H, CNCH), 13.48 (1H, OH).

The obtained products were analyzed using the following methods:

High-performance liquid chromatography with UV spectrophotometric and mass spectrometric detection (HPLC-UV-MS).

The method was used to determine the structure and molecular weight of obtained compounds, as well as quantitative composition and purity. Analysis was performed on an Agilent 1260 liquid chromatograph with sequential detection on photodiode-array (DAD) and mass-selective (MS) detectors. Separation was performed on an Agilent Zorbax Eclipse Plus C18 chromatographic column, using as eluent a 0.1% solution of formic acid in water and acetonitrile in a gradient elution mode. In the single-quadrupole mass spectrometric detector used, ionization was carried out by the electrospray method, and detection was performed in the mode of negative ion registration. Detection on a DAD was performed at a wavelength of 254 nm, and the UV spectrum was recorded in the range from 200 to 400 nm.

The enantiomeric composition of the product was determined on a Waters chromatograph equipped with a UV-detector. Separation was performed on a Chiralart Cellulose-SB chiral column manufactured by YMC, using as eluent a mixture of 10 mM potassium phosphate monosubstituted and acetonitrile in an isocratic elution mode.

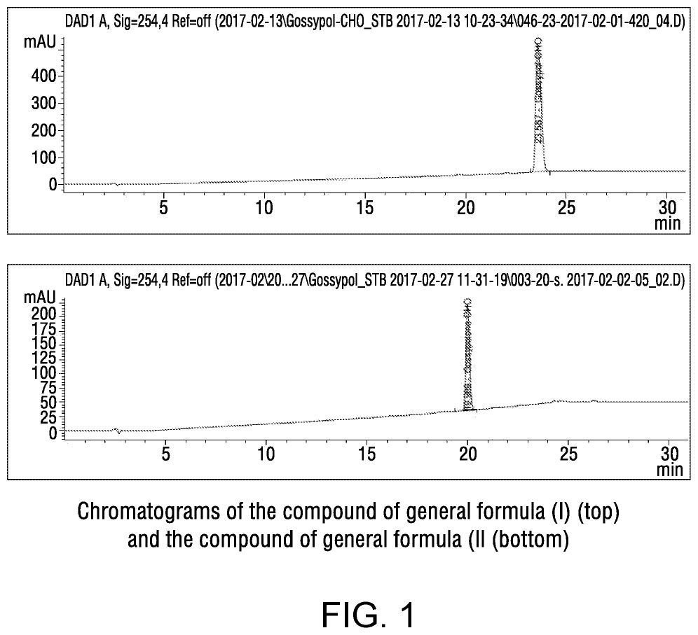

The purity of the obtained products was determined by HPLC. As shown in the represented chromatograms (FIG. 1), the isolated products I and II do not contain related impurities in significant quantities.

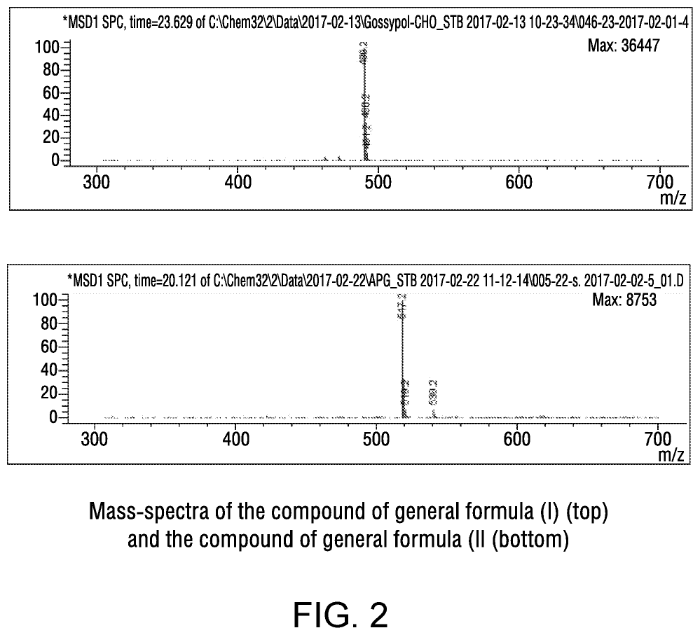

In the resultant mass spectrum of the main peak I with a retention time of 23.6 minutes, the principal ion is the pseudomolecular ion with m/z 489, which corresponds to the deprotonated molecule [M-H]—. In the mass spectrum of the main peak II with a retention time of 20.0 minutes, the primary molecular ion has m/z [M-H]—=517, and there is a fragment corresponding to the ion [M-2H+Na]13 with the mass of 539 (FIG. 2).

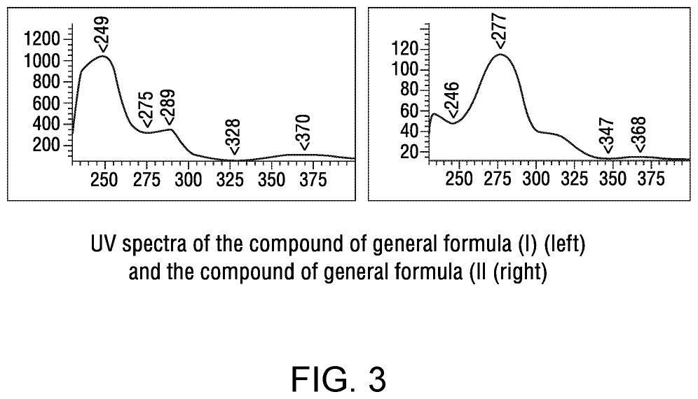

Compounds of the formulas I and II have characteristic UV spectra (FIG. 3). In the spectrum I, there are three peaks, at 249 nm, 289 nm and 370 nm. In the spectrum II, there is a single prominent maximum, at 277 nm.

As the result of the presence in molecule I of a C—C bond, which connects two naphthalene cycles and around which free rotation is hindered by bulk substituents, molecule I can exist as two enantiomers.

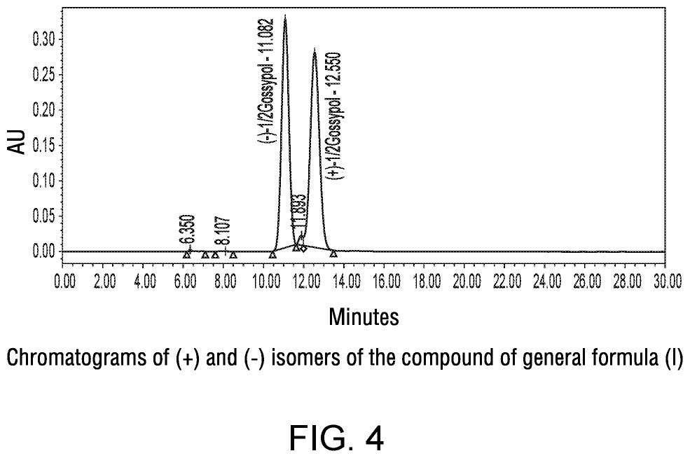

The chromatogram in FIG. 4 shows that the product is a racemic mixture consisting of (+) and (−) forms of molecule I, present in approximately equal amounts.

### Fourier-Transform Infrared Spectroscopy (FTIR)

FTIR spectroscopy was used to confirm the structure of the compounds obtained. The spectra were recorded on a Spectrum 65 spectrometer from Perkin Elmer in a disk with potassium bromide, in the range from 4000 to 400 cm-1.

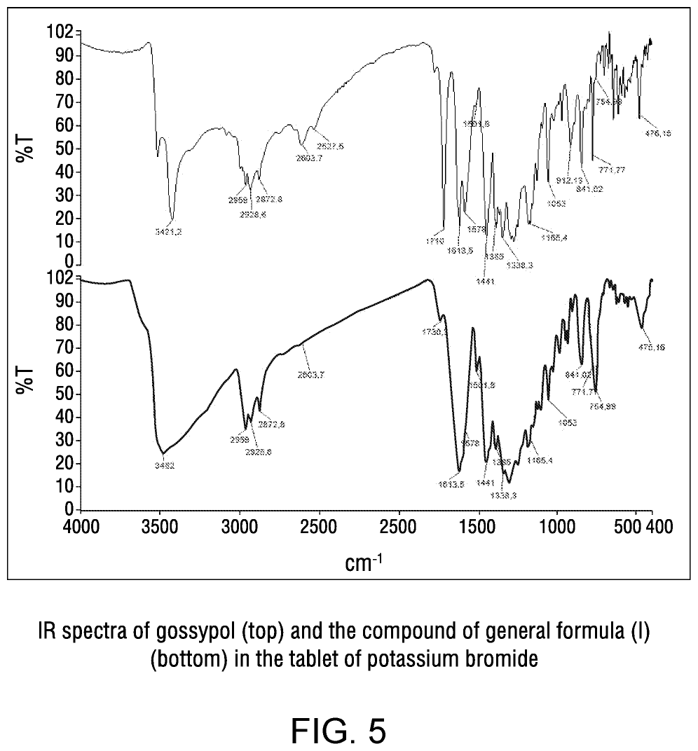

The FTIR spectrum I has characteristic bands for this structure (FIG. 5). In this spectrum, the signal at 3482 cm-1 corresponds to the valence vibrations of the OH groups of naphthalene rings. The group of signals in the range of 2960-2873 cm-1 corresponds to the vibrations of unsaturated and CH-groups of benzene rings and methyl groups CH3-. In contrast to the FTIR spectrum of gossypol, there is no pronounced band at the wave number of 2609 cm-1, corresponding to the valence vibrations of the CH fragment of aldehyde groups, and there is a shift and a significant decrease in the band intensity at 1711 cm-1 corresponding to the valence vibrations of C═O aldehyde groups. The bands at the wave numbers of 1614, 1578, and 1502 cm-1 correspond to the vibrations of carbon atoms at the double bond of naphthalene rings. The deformation vibrations of the methyl groups CH3- of the isopropyl fragment are represented by bands at 1441 and 1385 cm-1. The absorption bands at 1338 cm-1 and 1303 cm-1 correspond to the deformation vibrations of the naphthalene rings against each other, and the bands at 1122 cm-1 and 1053 cm-1—to the deformation vibrations of 1,2-substituted phenyls.

### Nuclear Magnetic Resonance Spectroscopy (NMR)

The 1H and 13C NMR spectra were recorded on a Bruker Avance-400 instrument (operating frequency 400 and 100 MHz, respectively). Chloroform signal was used as an internal standard (H=7.24, C=77.10 ppm).

Structures of molecules I and II were fully confirmed by 1H and 13C NMR spectroscopy data. In the 1H NMR spectra, there are all characteristic signals for these structures. In the spectrum of 1H II, there is a shift of the signal of the hydrogen atom in the aldehyde group, from δH=11.12 ppm to δH=10.53 ppm, and the signal forming an intramolecular hydrogen bond with it, of the hydrogen atom in a phenolic hydroxyl, from δH=15.15 ppm to δH=12.93 ppm, compared with the spectra of gossypol and molecule I. Compared with the gossypol spectrum, which has a singlet signal at δH=7.80 ppm, corresponding to two protons of the CH fragments of each of the naphthalene rings, three signals are observed for molecule I, at δH=7.45 ppm, δH=7.62 ppm and δH=7.74 ppm, which is due to the absence of one aldehyde group and the impaired molecule symmetry. In molecule II, there is one such proton, which manifests as a singlet with a chemical shift of 7.46 ppm. While NMR 1H of molecule I has a set of 5 singlet signals, corresponding to phenolic hydroxyls: δH=5.24 ppm, δH=5.86 ppm, δH=5.91 ppm, δH=5.98 ppm, δH=6.39 ppm, for molecule II there are three such signals: δH=6.23 ppm, δH=6.59 ppm and δH=6.93 ppm, which is fully consistent with the formulas of these compounds. In the 1H NMR spectra of both molecules in the strong field there are sets of signals corresponding to the isopropyl and methyl groups, which are slightly shifted to the weak field for molecule II.

Assessment of cytotoxic properties of asymmetric gossypol derivatives.

In 96-well flat-bottomed test plates, MDCK ECAAC (Sigma cat. [symbol] 85011435) cells are placed at 18,000 cells per well in a volume of 100 μl of complete medium containing 10% fetal calf serum (FCS). Cells are cultured for 24 hours in CO₂-incubator at 37° C. Then the liquid medium is removed from the wells of the test plates, leaving the immobilized living cells. Then wells are once washed with the 100 μl of serumless media. The medium is replaced with 100 μl per well of the dilutions of test compounds in support medium with 2% of serum, in 4 replicates. Each plate is prepared with 6 control wells containing 100 μl of fresh support medium without test compounds. The plates are incubated in CO₂-incubator at 37° C. for 48 hours. Then liquid contents are removed from the wells, and 100 μl of support medium and 20 μl of MTS (Promega, cat. [symbol] G3581) are added and mixed. The cells are incubated for 3 hours in CO₂-incubator at 37° C., and the relative optical density of the solution in each well is measured with a plate spectrophotometer at 492 nm against its absorption at a reference wavelength of 620 nm. The cytotoxicity (CC50) is calculated using the standard method.

An in vitro study of the antiviral effect of asymmetric gossypol derivatives on influenza A virus strain.

The experiments were performed at the same conditions as mentioned above (assessment of cytotoxic properties).

Serial dilutions of test compounds are added to MDCK cells that reached monolayer in 96-well culture plates and incubated in CO₂-incubator at 37° C. for 1 hour. After this, the cells are infected with 10-fold dilutions of the influenza virus A/Puerto Rico/8/34 (H1N1) in support medium. The plates are incubated in CO₂-incubator at 37° C. for 48 hours. As control, cells without test compounds are incubated at the same conditions. The virus titer values were evaluated by RGA with 0.75% of human erythrocytes in 0.9% NaCl solution. The exact values of IC50 and CC50 are determined by constructing non-linear regression of the data. The selectivity index is calculated by the formula SI=CC50/IC50.

TABLE 2

| | | CC50*, | IC50**, |
|---|---|---|---|
| | SI | mg/mL | mg/mL |
| Gossypol | 3,24 | 0,0040 | 0,0012 |
| II | 4,75 | 0,0038 | 0,0008 |
| I | 29,36 | 0,0055 | 0,0002 |

*CC50-toxic dose of the drug, causing the death of 50% of the monolayer cells.
**IC50-compound concentration, at which 50% of virus titer is inhibited.

1. A compound of general formula I 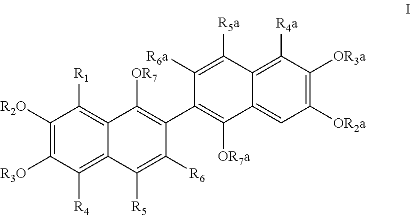
or general formula II 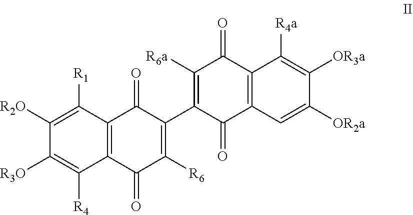
tautomeric forms, pharmaceutically acceptable salts and solvates thereof,
wherein:
R₁ represents: C(O)H, C═N−R₈, C═N−NH−R₈, C═N−NH−C(O)R₈;
R₈ represents: H; linear and branched C₁-C₂₀ alkyls, containing 0 to 10 substituents; linear and branched C₂-C₂₀ alkenyls and C₂-C₂₀ alkynyls with 1 to 9 double and (or) triple bonds, containing 0 to 10 substituents; unsubstituted and substituted cyclic systems with the number of atoms in the cycle of 3 to 20, containing 0 to 10 substituents, 0 to 10 double and (or) triple bonds, and 0 to 10 heteroatoms (N, S, O); unsubstituted and substituted aryls with 0 to 5 substituents; unsubstituted and substituted heteroaromatic compounds with the number of atoms in the cycle of 3 to 12 and the number of heteroatoms (N, S, O) of 1 to 6, containing 0 to 10 substituents; saturated, unsaturated and (or) aromatic polycyclic systems, containing 2 to 5 cycles with 3 to 8 atoms in the cycle, 0 to 10 double and (or) triple bonds and 0 to 10 heteroatoms (N, S, O), and 0 to 20 substituents;
R₂, R₃, R₂ₐ, R₃ₐ, R₇, R₇ₐ independently represent R₈ [symbol] or C(O)R₈;
R₅ [symbol] R₅ₐ independently represent H [symbol] or N═N—R₉;
wherein R₉ represents: unsaturated and saturated aryls with 0 to 5 substituents; unsaturated and saturated heteroaromatic compounds with the number of atoms in the cycle of 3 to 12 and the number of heteroatoms (N, S, O) of 1 to 6, containing 0 to 10 substituents; saturated, unsaturated and (or) aromatic polycyclic systems, containing 2 to 5 cycles with the number of atoms in the cycle of 3 to 8 and the number of heteroatoms of 0 to 10, and 0 to 20 substituents;
R₄, R₄ₐ, R₆ [symbol] R₆ₐ independently represent H, linear and branched C₁-C₁₀ alkyls;
substituents represent H, F, Cl, Br, I, NO₂, CN, SO₃H, SO₃M, SO₂Cl, CF₃, CCl₃, CBr₃, CI₃, C(O)H, COOH, COOM, C(O)Cl, C(O)Br, C(O)I, R₈, COOR₈, C(O)R₈, OH, OR₈, OC(O)R₈, OC(O)NR₈R₁₀, OSO₂R₈, NH₂, NR₈R₁₀, N⁺(R₈R₁₀R₁₁), NR₈C(O)R₁₀, NR₈SO₂R₁₀, NR₈C(O)NR₁₀R₁₁, SR₈, S(O)R₈, SO₂R₈, CH═NR₈;
R₁₀, R₁₁ independently represent: H; linear and branched C₁-C₂₀ alkyls, containing 0 to 10 substituents; linear and branched C₂-C₂₀ alkenyls and C₂-C₂₀ alkynyls with 1 to 9 double and (or) triple bonds, containing 0 to 10 substituents; unsaturated and saturated cyclic systems with the number of atoms in the cycle of 3 to 20, containing 0 to 10 substituents, with 0 to 10 double and (or) triple bonds and the number of heteroatoms (N, S, O) of 0 to 10; unsaturated and saturated aryls with 0 to 5 substituents, unsaturated and saturated heteroaromatic compounds with the number of atoms in the cycle of 3 to 12 and the number of heteroatoms (N, S, O) of 1 to 6, containing 0 to 10 substituents; saturated, unsaturated and (or) aromatic polycyclic systems, containing 2 to 5 cycles with the number of atoms in the cycle of 3 to 8, the number of double and (or) triple bonds of 0 to 10, and the number of heteroatoms (N, S, O) of 0 to 10, and 0 to 20 substituents;
M represents Li, Na, K, Cs, Rb.

2. A compound having the structure listed in Table 1:
TABLE 1

| | |
|---|---|
| I | 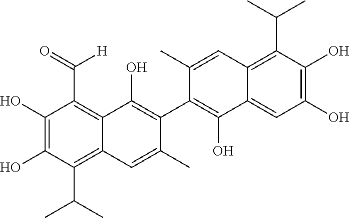 [SMILES: Cc1cc2c(C(C)C)c(O)c(O)cc2c(O)c1-c1c(C)cc2c(C(C)C)c(O)c(O)c(C=O)c2c1O] |
| II | 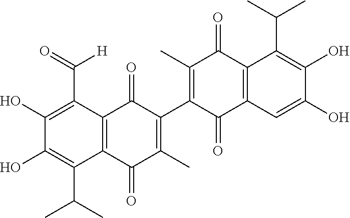 [SMILES: CC1=C(C2=C(C)C(=O)c3c(c(C=O)c(O)c(O)c3C(C)C)C2=O)C(=O)c2cc(O)c(O)c(C(C)C)c2C1=O] |
| III | 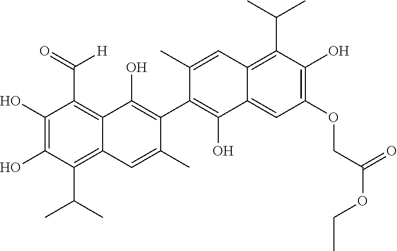 [SMILES: CCOC(=O)COc1cc2c(O)c(-c3c(C)cc4c(C(C)C)c(O)c(O)c(C=O)c4c3O)c(C)cc2c(C(C)C)c1O] |
| IV | 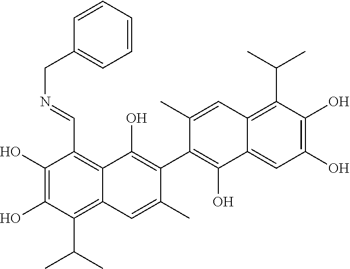 [SMILES: Cc1cc2c(C(C)C)c(O)c(O)cc2c(O)c1-c1c(C)cc2c(C(C)C)c(O)c(O)c(/C=N/Cc3ccccc3)c2c1O] |
| V | 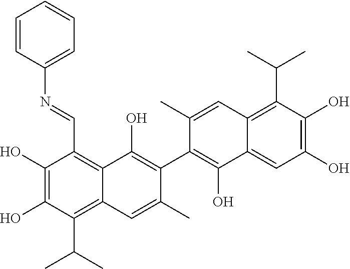 [SMILES: Cc1cc2c(C(C)C)c(O)c(O)cc2c(O)c1-c1c(C)cc2c(C(C)C)c(O)c(O)c(/C=N/c3ccccc3)c2c1O] |
| VI | 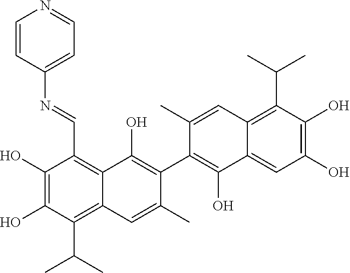 [SMILES: Cc1cc2c(C(C)C)c(O)c(O)cc2c(O)c1-c1c(C)cc2c(C(C)C)c(O)c(O)c(/C=N/c3ccncc3)c2c1O] |
| VII | 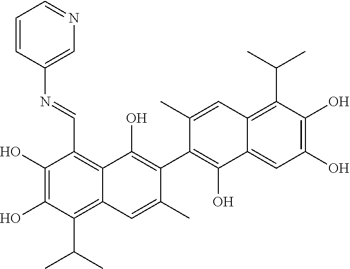 [SMILES: Cc1cc2c(C(C)C)c(O)c(O)cc2c(O)c1-c1c(C)cc2c(C(C)C)c(O)c(O)c(/C=N/c3cccnc3)c2c1O] |
| VIII | 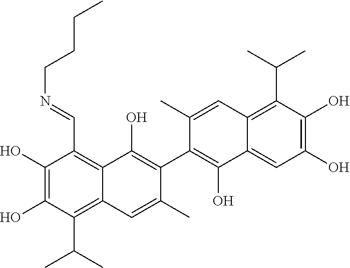 [SMILES: CCCC/N=C/c1c(O)c(O)c(C(C)C)c2cc(C)c(-c3c(C)cc4c(C(C)C)c(O)c(O)cc4c3O)c(O)c12] |
| IX | 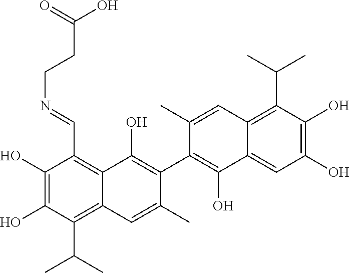 [SMILES: Cc1cc2c(C(C)C)c(O)c(O)cc2c(O)c1-c1c(C)cc2c(C(C)C)c(O)c(O)c(/C=N/CCC(=O)O)c2c1O] |
| X | 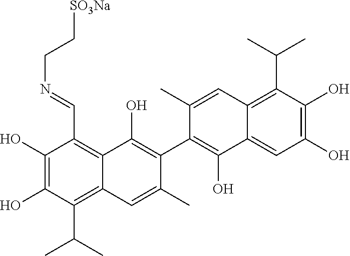 |
| XI | 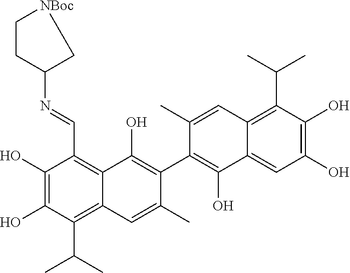 [SMILES: Cc1cc2c(C(C)C)c(O)c(O)cc2c(O)c1-c1c(C)cc2c(C(C)C)c(O)c(O)c(/C=N/C3CCN(C(=O)OC(C)(C)C)C3)c2c1O] |
| XII | 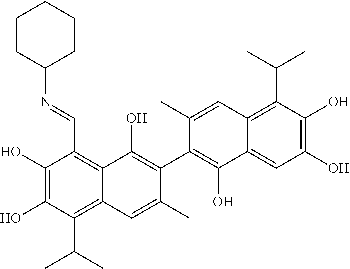 [SMILES: Cc1cc2c(C(C)C)c(O)c(O)cc2c(O)c1-c1c(C)cc2c(C(C)C)c(O)c(O)c(/C=N/C3CCCCC3)c2c1O] |
| XIII | 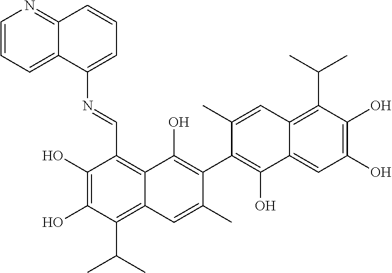 [SMILES: Cc1cc2c(C(C)C)c(O)c(O)cc2c(O)c1-c1c(C)cc2c(C(C)C)c(O)c(O)c(/C=N/c3cccc4ncccc34)c2c1O] |
| XIV | 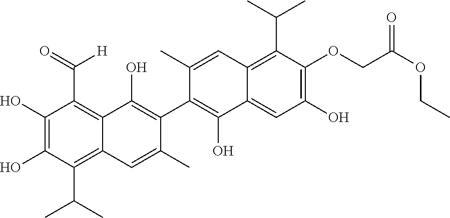 [SMILES: CCOC(=O)COc1c(O)cc2c(O)c(-c3c(C)cc4c(C(C)C)c(O)c(O)c(C=O)c4c3O)c(C)cc2c1C(C)C] |
| XV | 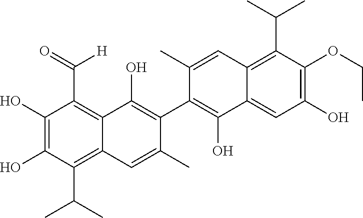 [SMILES: CCOc1c(O)cc2c(O)c(-c3c(C)cc4c(C(C)C)c(O)c(O)c(C=O)c4c3O)c(C)cc2c1C(C)C] |
| XVI | 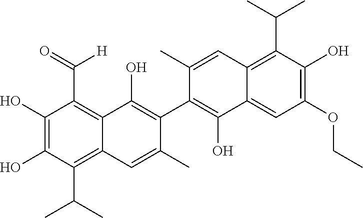 [SMILES: CCOc1cc2c(O)c(-c3c(C)cc4c(C(C)C)c(O)c(O)c(C=O)c4c3O)c(C)cc2c(C(C)C)c1O] |
| XVII | 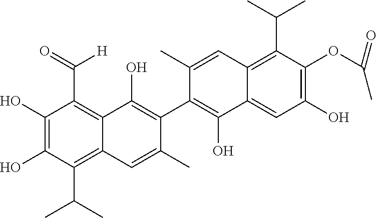 [SMILES: CC(=O)Oc1c(O)cc2c(O)c(-c3c(C)cc4c(C(C)C)c(O)c(O)c(C=O)c4c3O)c(C)cc2c1C(C)C] |
| XVIII | 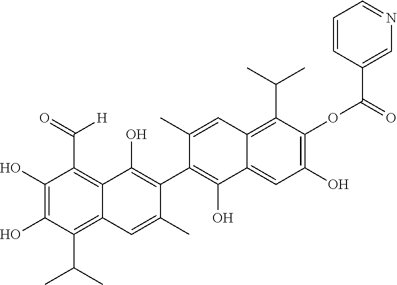 [SMILES: Cc1cc2c(C(C)C)c(OC(=O)c3cccnc3)c(O)cc2c(O)c1-c1c(C)cc2c(C(C)C)c(O)c(O)c(C=O)c2c1O] |
| XIX | 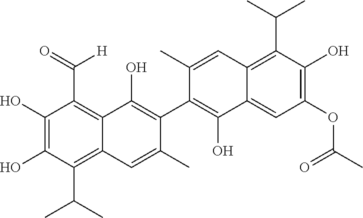 [SMILES: CC(=O)Oc1cc2c(O)c(-c3c(C)cc4c(C(C)C)c(O)c(O)c(C=O)c4c3O)c(C)cc2c(C(C)C)c1O] |
| XX | 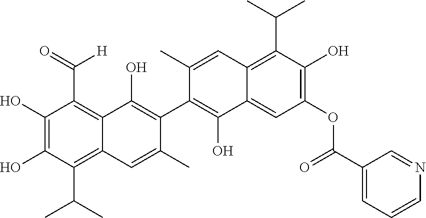 [SMILES: Cc1cc2c(C(C)C)c(O)c(OC(=O)c3cccnc3)cc2c(O)c1-c1c(C)cc2c(C(C)C)c(O)c(O)c(C=O)c2c1O] |
| XXI | 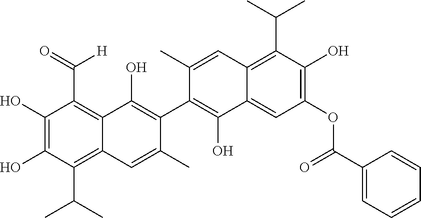 [SMILES: Cc1cc2c(C(C)C)c(O)c(OC(=O)c3ccccc3)cc2c(O)c1-c1c(C)cc2c(C(C)C)c(O)c(O)c(C=O)c2c1O] |
| XXII | 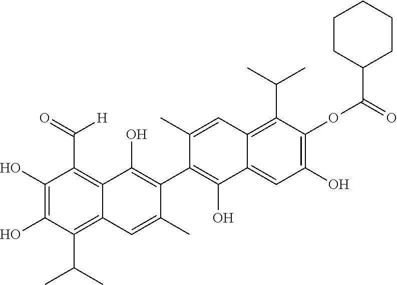 [SMILES: Cc1cc2c(C(C)C)c(OC(=O)C3CCCCC3)c(O)cc2c(O)c1-c1c(C)cc2c(C(C)C)c(O)c(O)c(C=O)c2c1O] |
| XXIII | 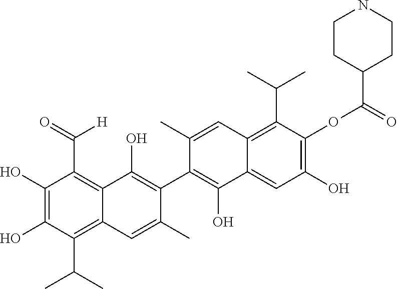 [SMILES: Cc1cc2c(C(C)C)c(OC(=O)C3CCNCC3)c(O)cc2c(O)c1-c1c(C)cc2c(C(C)C)c(O)c(O)c(C=O)c2c1O] |
| XXIV | 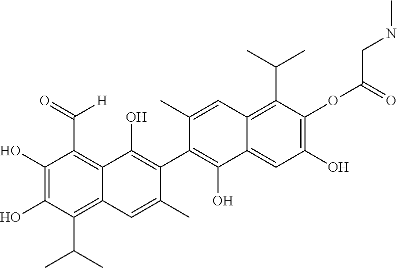 [SMILES: CNCC(=O)Oc1c(O)cc2c(O)c(-c3c(C)cc4c(C(C)C)c(O)c(O)c(C=O)c4c3O)c(C)cc2c1C(C)C] |
| XXV | 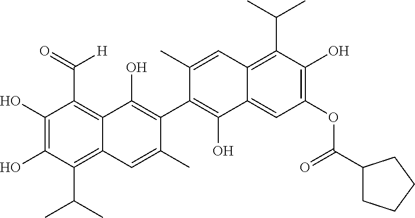 [SMILES: Cc1cc2c(C(C)C)c(O)c(OC(=O)C3CCCC3)cc2c(O)c1-c1c(C)cc2c(C(C)C)c(O)c(O)c(C=O)c2c1O] |
| XXVI | 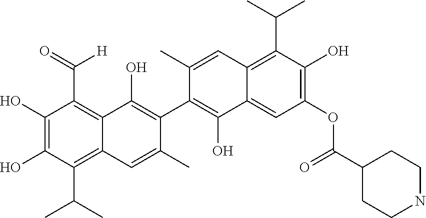 [SMILES: Cc1cc2c(C(C)C)c(O)c(OC(=O)C3CCNCC3)cc2c(O)c1-c1c(C)cc2c(C(C)C)c(O)c(O)c(C=O)c2c1O] |
| XXVII | 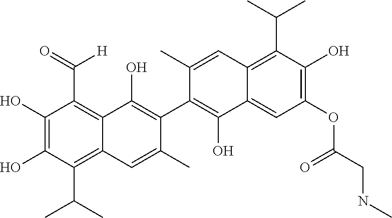 [SMILES: CNCC(=O)Oc1cc2c(O)c(-c3c(C)cc4c(C(C)C)c(O)c(O)c(C=O)c4c3O)c(C)cc2c(C(C)C)c1O] |
| XXVIII | 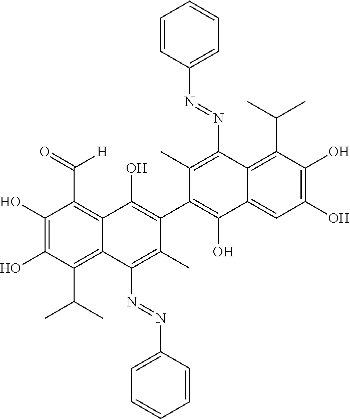 [SMILES: Cc1c(-c2c(C)c(/N=N/c3ccccc3)c3c(C(C)C)c(O)c(O)c(C=O)c3c2O)c(O)c2cc(O)c(O)c(C(C)C)c2c1/N=N/c1ccccc1] |
| XXIX | 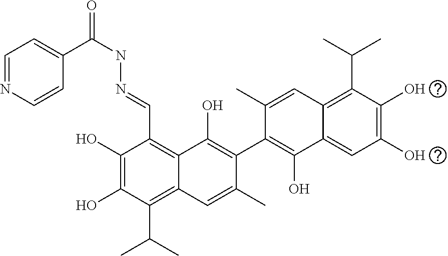 [SMILES: Cc1cc2c(C(C)C)c(O)c(O)cc2c(O)c1-c1c(C)cc2c(C(C)C)c(O)c(O)c(/C=N/NC(=O)c3ccncc3)c2c1O] |
| XXX | 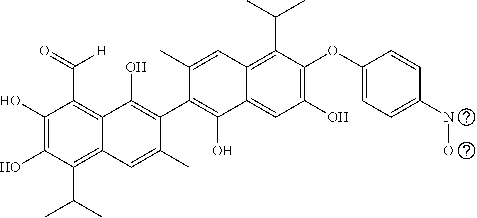 [SMILES: Cc1cc2c(C(C)C)c(Oc3ccc(NO)cc3)c(O)cc2c(O)c1-c1c(C)cc2c(C(C)C)c(O)c(O)c(C=O)c2c1O] |
| XXXI | 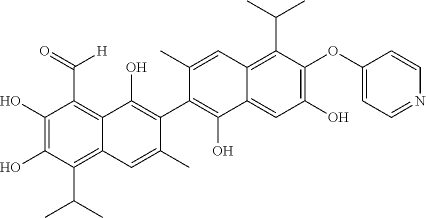 [SMILES: Cc1cc2c(C(C)C)c(Oc3ccncc3)c(O)cc2c(O)c1-c1c(C)cc2c(C(C)C)c(O)c(O)c(C=O)c2c1O] |
| XXXII | 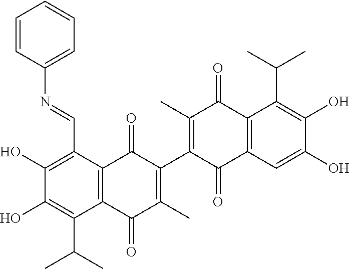 [SMILES: CC1=C(C2=C(C)C(=O)c3c(c(/C=N/c4ccccc4)c(O)c(O)c3C(C)C)C2=O)C(=O)c2cc(O)c(O)c(C(C)C)c2C1=O] |
| XXXIII | 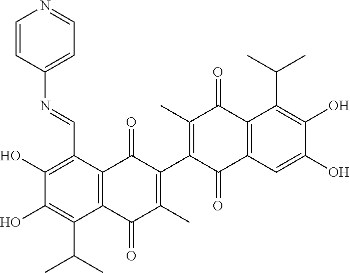 [SMILES: CC1=C(C2=C(C)C(=O)c3c(c(/C=N/c4ccncc4)c(O)c(O)c3C(C)C)C2=O)C(=O)c2cc(O)c(O)c(C(C)C)c2C1=O] |
| XXXIV | 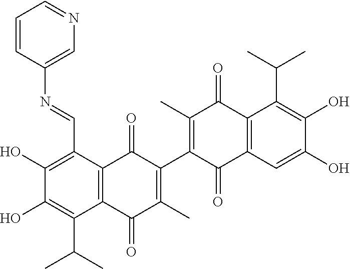 [SMILES: CC1=C(C2=C(C)C(=O)c3c(c(/C=N/c4cccnc4)c(O)c(O)c3C(C)C)C2=O)C(=O)c2cc(O)c(O)c(C(C)C)c2C1=O] |
| XXXV | 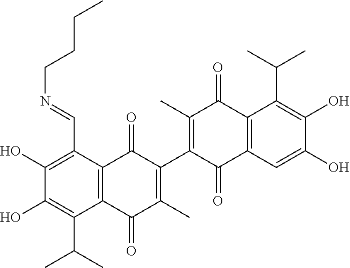 [SMILES: CCCC/N=C/c1c(O)c(O)c(C(C)C)c2c1C(=O)C(C1=C(C)C(=O)c3c(cc(O)c(O)c3C(C)C)C1=O)=C(C)C2=O] |
| XXXVI | 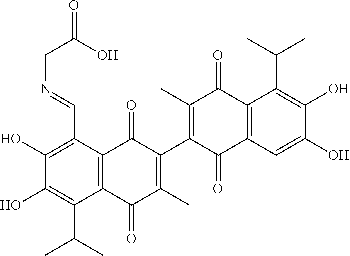 [SMILES: CC1=C(C2=C(C)C(=O)c3c(c(/C=N/CC(=O)O)c(O)c(O)c3C(C)C)C2=O)C(=O)c2cc(O)c(O)c(C(C)C)c2C1=O] |
| XXXVII | 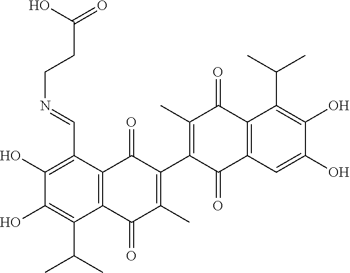 [SMILES: CC1=C(C2=C(C)C(=O)c3c(c(/C=N/CCC(=O)O)c(O)c(O)c3C(C)C)C2=O)C(=O)c2cc(O)c(O)c(C(C)C)c2C1=O] |
| XXXVIII | 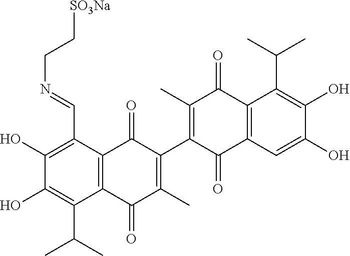 |
| XXXIX | 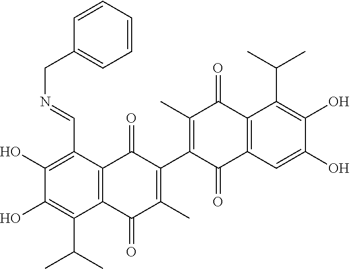 [SMILES: CC1=C(C2=C(C)C(=O)c3c(c(/C=N/Cc4ccccc4)c(O)c(O)c3C(C)C)C2=O)C(=O)c2cc(O)c(O)c(C(C)C)c2C1=O] |
| XL | 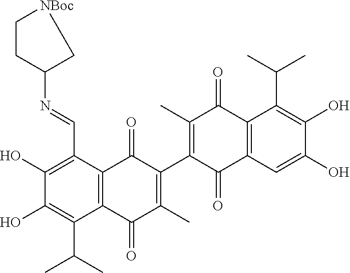 [SMILES: CC1=C(C2=C(C)C(=O)c3c(c(/C=N/C4CCN(C(=O)OC(C)(C)C)C4)c(O)c(O)c3C(C)C)C2=O)C(=O)c2cc(O)c(O)c(C(C)C)c2C1=O] |
| XL1 | 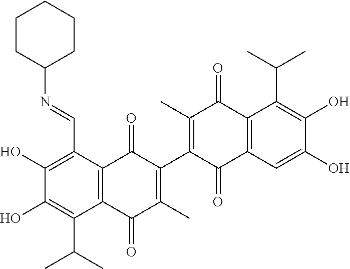 [SMILES: CC1=C(C2=C(C)C(=O)c3c(c(/C=N/C4CCCCC4)c(O)c(O)c3C(C)C)C2=O)C(=O)c2cc(O)c(O)c(C(C)C)c2C1=O] |
| XLII | 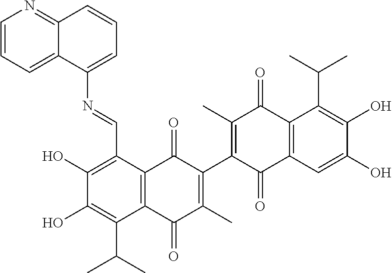 [SMILES: CC1=C(C2=C(C)C(=O)c3c(c(/C=N/c4cccc5ncccc45)c(O)c(O)c3C(C)C)C2=O)C(=O)c2cc(O)c(O)c(C(C)C)c2C1=O] |
| XLIII | 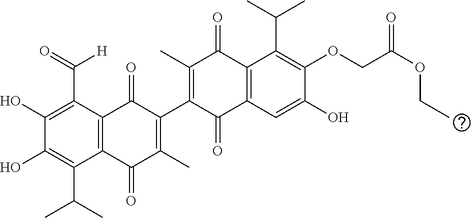 [SMILES: CCOC(=O)COc1c(O)cc2c(c1C(C)C)C(=O)C(C)=C(C1=C(C)C(=O)c3c(c(C=O)c(O)c(O)c3C(C)C)C1=O)C2=O] |
| XLIV | 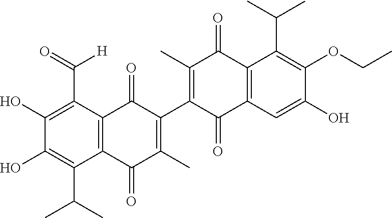 [SMILES: CCOc1c(O)cc2c(c1C(C)C)C(=O)C(C)=C(C1=C(C)C(=O)c3c(c(C=O)c(O)c(O)c3C(C)C)C1=O)C2=O] |
| XLV | 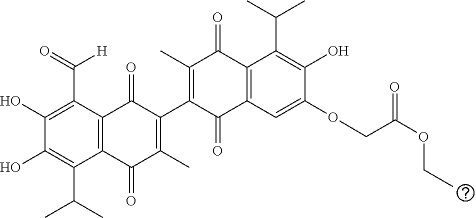 [SMILES: CCOC(=O)COc1cc2c(c(C(C)C)c1O)C(=O)C(C)=C(C1=C(C)C(=O)c3c(c(C=O)c(O)c(O)c3C(C)C)C1=O)C2=O] |
| XLVI | 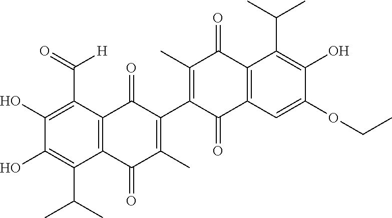 [SMILES: CCOc1cc2c(c(C(C)C)c1O)C(=O)C(C)=C(C1=C(C)C(=O)c3c(c(C=O)c(O)c(O)c3C(C)C)C1=O)C2=O] |
| XLVII | 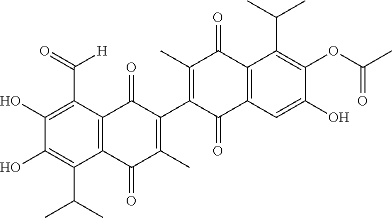 [SMILES: CC(=O)Oc1c(O)cc2c(c1C(C)C)C(=O)C(C)=C(C1=C(C)C(=O)c3c(c(C=O)c(O)c(O)c3C(C)C)C1=O)C2=O] |
| XLVIII | 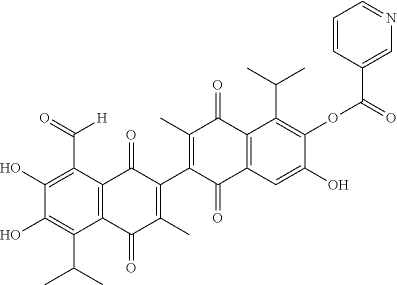 [SMILES: CC1=C(C2=C(C)C(=O)c3c(c(C=O)c(O)c(O)c3C(C)C)C2=O)C(=O)c2cc(O)c(OC(=O)c3cccnc3)c(C(C)C)c2C1=O] |
| XLIX | 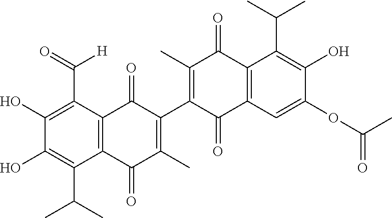 [SMILES: CC(=O)Oc1cc2c(c(C(C)C)c1O)C(=O)C(C)=C(C1=C(C)C(=O)c3c(c(C=O)c(O)c(O)c3C(C)C)C1=O)C2=O] |
| L | 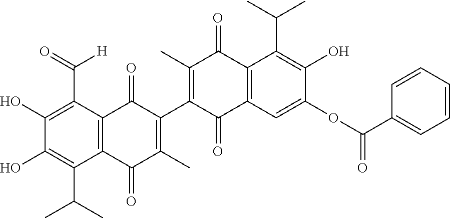 [SMILES: CC1=C(C2=C(C)C(=O)c3c(c(C=O)c(O)c(O)c3C(C)C)C2=O)C(=O)c2cc(OC(=O)c3ccccc3)c(O)c(C(C)C)c2C1=O] |
| LI | 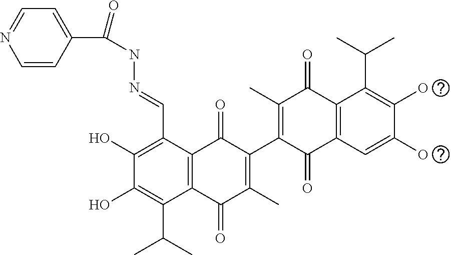 [SMILES: CC1=C(C2=C(C)C(=O)c3c(c(/C=N/NC(=O)c4ccncc4)c(O)c(O)c3C(C)C)C2=O)C(=O)c2cc(O)c(O)c(C(C)C)c2C1=O] |
| LII | 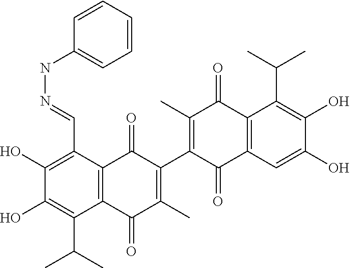 [SMILES: CC1=C(C2=C(C)C(=O)c3c(c(/C=N/Nc4ccccc4)c(O)c(O)c3C(C)C)C2=O)C(=O)c2cc(O)c(O)c(C(C)C)c2C1=O] |
| LIII | 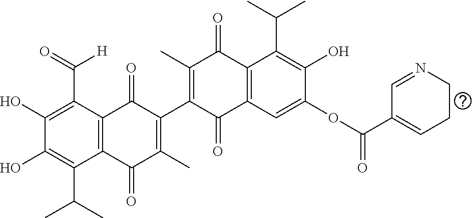 [SMILES: CC1=C(C2=C(C)C(=O)c3c(c(C=O)c(O)c(O)c3C(C)C)C2=O)C(=O)c2cc(OC(=O)C3=CCCN=C3)c(O)c(C(C)C)c2C1=O] |
| LIV | 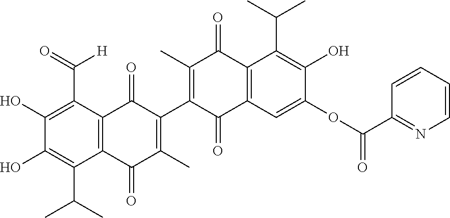 [SMILES: CC1=C(C2=C(C)C(=O)c3c(c(C=O)c(O)c(O)c3C(C)C)C2=O)C(=O)c2cc(OC(=O)c3ccccn3)c(O)c(C(C)C)c2C1=O] |
| LV | 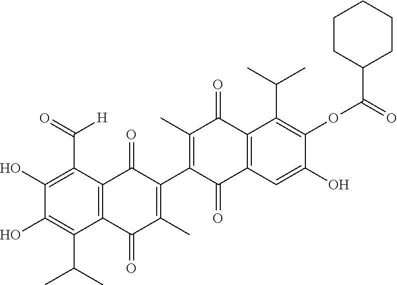 [SMILES: CC1=C(C2=C(C)C(=O)c3c(c(C=O)c(O)c(O)c3C(C)C)C2=O)C(=O)c2cc(O)c(OC(=O)C3CCCCC3)c(C(C)C)c2C1=O] |
| LVI | 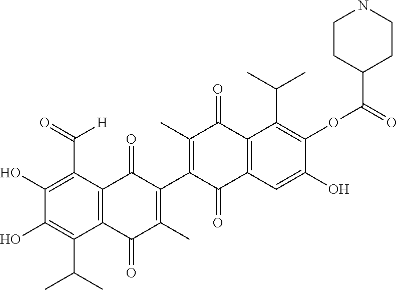 [SMILES: CC1=C(C2=C(C)C(=O)c3c(c(C=O)c(O)c(O)c3C(C)C)C2=O)C(=O)c2cc(O)c(OC(=O)C3CCNCC3)c(C(C)C)c2C1=O] |
| LVII | 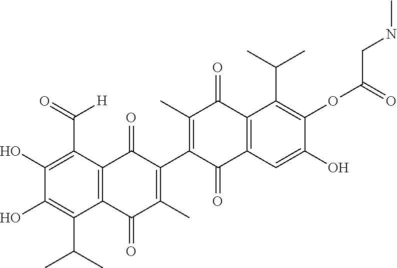 [SMILES: CNCC(=O)Oc1c(O)cc2c(c1C(C)C)C(=O)C(C)=C(C1=C(C)C(=O)c3c(c(C=O)c(O)c(O)c3C(C)C)C1=O)C2=O] |
| LVIII | 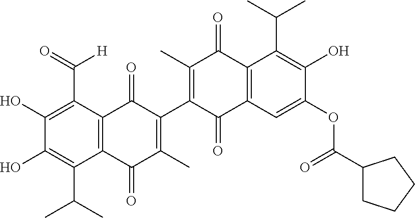 [SMILES: CC1=C(C2=C(C)C(=O)c3c(c(C=O)c(O)c(O)c3C(C)C)C2=O)C(=O)c2cc(OC(=O)C3CCCC3)c(O)c(C(C)C)c2C1=O] |
| LIX | 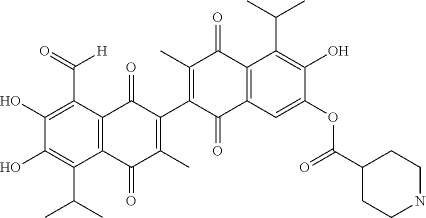 [SMILES: CC1=C(C2=C(C)C(=O)c3c(c(C=O)c(O)c(O)c3C(C)C)C2=O)C(=O)c2cc(OC(=O)C3CCNCC3)c(O)c(C(C)C)c2C1=O] |
| LX | 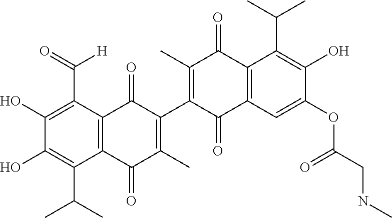 [SMILES: CNCC(=O)Oc1cc2c(c(C(C)C)c1O)C(=O)C(C)=C(C1=C(C)C(=O)c3c(c(C=O)c(O)c(O)c3C(C)C)C1=O)C2=O] |
| LXI | 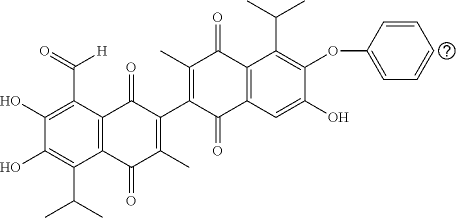 [SMILES: CC1=C(C2=C(C)C(=O)c3c(c(C=O)c(O)c(O)c3C(C)C)C2=O)C(=O)c2cc(O)c(Oc3ccccc3)c(C(C)C)c2C1=O] |
| LXII | 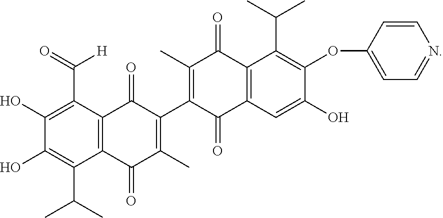 [SMILES: CC1=C(C2=C(C)C(=O)c3c(c(C=O)c(O)c(O)c3C(C)C)C2=O)C(=O)c2cc(O)c(Oc3ccncc3)c(C(C)C)c2C1=O] |

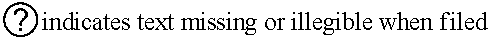

3. A process of preparing a compound of general formula I according to claim 1, comprising the following stages:
treatment of the gossypol derivative, optionally in its solvate form, with an aqueous solution of alkali or alkaline earth metal hydroxide;
treatment of the resultant reaction mixture with organic and/or inorganic acids to pH=0.1-10, preferably 3-5;
the reaction product isolation and purification;
and optionally subsequent treatment of resultant product with a reagent, which is selected from a group containing: aliphatic, aromatic or heteroaromatic amines; alkylating agents, acylating agents; diazonium salts; H and
target product isolation and purification.

4. The process according to claim 3, wherein the gossypol derivative represents gossypol, gossypol acetic acid, methyl ethers of gossypol.

5. (canceled)

6. The process according to claim 3, wherein the concentration of the aqueous solution of alkali or alkaline earth metal hydroxide, preferably sodium hydroxide or potassium hydroxide, is 0.1-50 mass %, preferably 10-30 mass %.

7. The process according to claim 3, wherein treatment with aliphatic, aromatic or heteroaromatic amines is performed in solvent, such as isopropanol, diethyl ether, dioxane or tetrahydrofuran.

8. The process according to claim 3, wherein aromatic or aliphatic halogen derivatives or alkyl sulfates are used as alkylating agents, and treatment is performed in the presence of organic and/or inorganic bases, such as cesium carbonate, sodium ethylate, potassium tert-butylate, triethylamine or sodium hydride, in a solvent, such as isopropanol, diethyl ester or tetrahydrofuran.

9. The process according to claim 3, wherein carbonic acids or halogen anhydrides are used as an acylating agent, and treatment is performed in the presence of organic and/or inorganic bases, such as potassium carbonate, cesium carbonate, sodium methylate, sodium ethylate, potassium tert-butylate, triethylamine, diisopropylamine or sodium hydride, optionally in the presence of coupling reagents such as carbonyldiimidazol, DCC, in a solvent such as isopropanol, diethyl ether or tetrahydrofuran.

10. The process according to claim 3, wherein treatment with diazonium salts is performed in the aqueous environment and/or in solvent, such as ethanol or diethyl ether.

11. The process of preparing a compound of general formula (II) according to claim 1, comprising the following stages:
treatment of gossypol derivative, optionally in the form of its solvate, with an aqueous solution of alkali and/or alkaline earth metal hydroxide;
treatment of the reaction mixture with organic and/or inorganic acids to pH=0.1-10, preferably 3-5;
the resultant product isolation and purification;
subsequent treatment of the resultant product with hydrogen peroxide at the concentration of 0.1-99.8% in organic and/or inorganic acids; or
subsequent treatment of the resultant product with trivalent ferric compounds in the presence of an organic solvent and/or an organic or inorganic acid;
the resultant product isolation and purification; and
optionally, subsequent treatment of resultant product with a reagent selected from a group containing: aliphatic, aromatic or heteroaromatic amines; alkylating agents and acylating agents; and
target product isolation and purification.

12. The process according to claim 11, wherein gossypol derivative represents gossypol, gossypol acetic acid, gossypol methyl ester.

13. (canceled)

14. The process according to claim 11, wherein the concentration of the aqueous solution of alkali or alkaline earth metal hydroxide, preferably potassium hydroxide or sodium hydroxide, is 0.1-50 mass %, preferably 10-30 mass %.

15. The process according to claim 11, wherein hydrogen peroxide is used at the concentration of 1-30 mass % and treatment is performed in the acetic acid environment.

16. The process according to claim 11, wherein trivalent ferric compound represents ferric chloride (III) in the solid form or in an aqueous solution, and treatment is performed in the acetone and/or acetic acid environment.

17. The process according to claim 11, wherein treatment with aliphatic, aromatic or heteroaromatic amines is performed in a solvent, such as isopropanol, diethyl ether, dioxane or tetrahydrofuran.

18. The process according to claim 11, wherein aromatic or aliphatic halogen derivatives or alkyl sulfates are used as alkylating agents, and treatment is performed in the presence of organic and/or inorganic bases, such as cesium carbonate, sodium ethylate, potassium tert-butylate, triethylamine or sodium hydride, in a solvent, such as isopropanol, diethyl ether or tetrahydrofuran.

19. The process according to claim 11, wherein carbonic acids or halogen anhydrides are used as an acylating agent, and treatment is performed in the presence of organic and/or inorganic bases, such as potassium carbonate, cesium carbonate, sodium methylate, sodium ethylate, potassium tert-butylate, triethylamine, diisopropylamine or sodium hydride, not necessarily in the presence of coupling reagents such as carbonyldiimidazol, DCC, in a solvent such as chloroform, tetrahydrofuran or dioxane.

20. An antiviral agent, which represents a compound of general formula (I) or (II) according to claim 1.

21. The antiviral agent according to claim 20, effective against influenza, herpes, hepatitis, HIV viruses.
